(19) United States
(12) Patent Application Publication
(10) Pub. No.: US 2025/0009631 A1
(43) Pub. Date: Jan. 9, 2025

(54) METHOD OF NAIL STRENGTHENING WITH A COMPLEX MICHAEL ADDITION TREATMENT COMPOSITION

(71) Applicant: Wella Operations US, LLC, Calabasas, CA (US)

(72) Inventors: Zhiwei Xie, Calabasas, CA (US); Laurisa London, Calabasas, CA (US); Sunan Yuvavanich, Calabasas, CA (US)

(21) Appl. No.: 18/813,311

(22) Filed: Aug. 23, 2024

Related U.S. Application Data
(63) Continuation-in-part of application No. PCT/US2023/065266, filed on Apr. 3, 2023.
(60) Provisional application No. 63/578,725, filed on Aug. 25, 2023.

(51) Int. Cl. A61K 8/58; A61K 8/44; A61Q 3/02

(52) U.S. Cl. CPC A61K 8/585; A61K 8/44; A61Q 3/02

(57) ABSTRACT

A method for treatment of brittle, broken, cracked or otherwise damaged nail plates. The method uses a treatment composition of an aqueous-organic medium containing a Michael addition monoadduct or bisadduct of complex diamine and maleic anhydride.

### CLAIM OF PRIORITY

This patent application claims continuation in part benefit from PCT Application Serial No. PCT/US23/65266 filed Apr. 3, 2023 and claims the benefit of priority from U.S. Provisional Patent Application Ser. No. 63/578,725 filed Aug. 25, 2023, the disclosures of which are incorporated herein in their entirety.

### FIELD OF INVENTION

The present invention relates to a method for treating a damaged nail plate using a Michael addition treatment composition.

### BACKGROUND

Human digital soft tissue serves as one of the primary sensory interactions with a human environment. For this reason, digital soft tissue is so sensitive to tactile, heat, cold and electromagnetic (infrared and UV light) radiation that too much can cause injury. The nail plates protect this digital tissue and the corresponding internal bone from assault and shock which would otherwise occur by heating, freezing, irradiating, touching, gripping, grasping, walking and/or standing with or on the digital appendages. For example, human fingernails absorb and repel shock to the soft tissue and bone when the end of a finger is slammed against a hard object. A nail plate that is weak, brittle, cracked and/or broken does not protect against this shock so that the assault results in significant pain.

Traditionally, broken, cracked or weak nail plates have been treated with a formaldehyde composition to mend the breaks and cracks. The formaldehyde reacts with the proteins of the nail to form imine bonds and harden the tissue. Not only does the treatment include a strong odor, but also kills associated cells and often causes allergic reaction. The treatment is the same as is performed by embalming techniques.

Nevertheless, formaldehyde treatment of cracked and broken nails is the currently approved FDA treatment. Recently, other aldehyde formulations have been developed in attempts to circumvent the undesirable side effects of formaldehyde treatment. Citral, lemonal/geranial and citronellal are aldehydic terpenoids traditionally used in minute amounts for aroma development in perfumes. Recently, citral and citronellal formulations having higher concentrations of this aldehyde have been developed for use in nail strengthening applications. The difficulties associated with formaldehyde also are problems for these longer carbon chain aldehydes.

Other nail strengthening treatments include iodine, hydrazines, divinyl sulfone and dithiopyridine. Of these, iodine is the active ingredient in some commercial nail strengtheners while hydrazines, divinyl sulfone and dithiopyridine are not regarded as safe alternatives to formaldehyde. Bismaleimidohexane and bismaleimidotriethylene glycol are also agents known for the treatment of brittle, frizzy hair. They might also be useful as well for strengthening nails since nails and hair include similar families of proteins.

More recently, a nail strengthening treatment using a non-reacted, non-covalent complex of a diamine and an unsaturated dicarboxylic acid such as maleic acid has been described in US Published Application 2022/0257534 and PCT/US2018/041408 filed Jul. 10, 2018.

Therefore, it continues to be desirable to develop a safe, effective, non-offensive treatment for weak, broken, cracked nail plates that provides as strong as possible strengthening and reformation of broken nails. Another goal includes the development of a formulation that does not cause discoloration of a human nail plate and that can optionally be combined with color components for coloring a nail plate.

### SUMMARY OF THE INVENTION

The present invention is directed to method for treatment of a fingernail or toenail that is weak, brittle, inflexible, cracked and/or broken through application to the nail plate of concern of the finger or toe a nail strengthening composition. Embodiments of the method of treatment apply to a damaged nail plate a treatment composition of an organic or aqueous or aqueous-organic medium in combination with a Michael addition product comprising a monoadduct which is a 2-(multi-organo- or siliconyl-amino)succinic acid, ester, amide, anhydride or imide and/or a bisadduct which is an N,N′-bis-(2-succinoylhydroxy)-, or N,N′-bis-(2-succinoyloxy alkylester)-, or, N,N′-bis(2-succinamido)-, or N,N′-bis(2-succinanhydrido)-, or N,N′-bis(2-succinimido)-, α,ω-multi-organo- or siliconyl-diamine or any combination thereof.

The monoadduct and/or the bisadduct may be prepared by combining, through a Michael addition reaction, an α, β unsaturated carbonyl compound such as maleic acid or maleic diester or maleic diamide or maleic anhydride or maleimide with a diamine such as a multi-organo diamine or a silicone diamine. The multi-organo diamine has primary amine groups at its termini and the multi-organo moiety between the termini may be an linear or branched, preferably linear, organic chain composed of such backbone chains as a long chain hydrocarbon, a hetero-hydrocarbon chain with multiple nitrogen, oxygen and/or sulfur atoms interspersed in the chain, a polyamide chain, a polyester chain, a hydrocarbon or hetero-hydrocarbon or polyamide or polyester chain having pendant and/or terminal ester, amide, and/or amine groups, a cycloaliphatic chain or an aromatic chain. The silicone diamine has primary amines at its termini and/or at one or more branch chains and the silicone chain between the termini may be a linear silicone polymer chain composed of dimethyl siloxane moieties and/or a block copolymer chain of dimethylsiloxane polymer blocks and polyether, polyester and/or polyamide blocks. Typically, the amine groups are configured as aminoalkyl or aminoalkylenyl-mono-, di- or tri-iminoalkylenyl groups.

Embodiments of the treatment composition may be prepared according to known procedures through use of solventless or solvent reaction conditions with heat and optional reaction promotor for efficient hydrogen transfer. The Michael addition reaction products may be isolated and purified to provide the monoadduct, the bisadduct or a combination thereof. It has been discovered that an alternative method for preparation of the treatment composition may be accomplished by forming an alcoholic dispersion or solution of the starting materials and allowing the dispersion or solution to rest at ambient temperature for an extended period such as up to six to twelve months. The resulting mixture has been found to contain the monoadduct and/or bisadduct with unreacted starting materials. The resulting mixture may be purified to provide monoadduct and/or bisadduct or may be used as a treatment composition without further purification.

Embodiments of the treatment composition also optionally include the multi-organo- or siliconyl-diamine starting compound optionally with the α, β unsaturated carbonyl starting compound in combination with the monoadduct and/or bisadduct as the treatment composition for nail strengthening. In at least some instances of the Michael addition reaction, a minor to major amount of the diamine starting compound remains as a component of the treatment composition. The diamine starting material also can have nail strengthening ability. Consequently, in such situations, the diamine may remain with the mono and/or bis adduct.

Embodiments of the treatment composition incorporating the monoadduct, the bisadduct, their mixture, at least some of the multi-organo or siliconyl-diamine and any combination thereof provide multifunctional use properties including but not limited to desirable aqueous and dissolution ability, desirable nail surface penetration and desirable intermolecular binding of keratin proteins within damaged regions of broken and/or cracked nails.

The acid and/or base salts of the mono and/or bis adducts and the acid salts of the multi-organo- or siliconyl-diamine as well as pH control of the medium are additional aspects of these embodiments of the invention.

When embodiments of the treatment composition constitute the monoadduct alone, the bisadduct alone or a mixture of the monoadduct and bisadduct and/or their salts, any amount of monoadduct or bisadduct or mixture or salts may be used in the treatment composition up to a weight percent saturation of monoadduct or bisadduct or mixture in the aqueous or aqueous organic medium (for example up to as much as 40 to 60 wt % or more depending upon the substituents of the mono and bis adducts and the pH of the medium). Exemplary ratios of the monoadduct to bisadduct in a mixture may range from almost indetectable to essentially pure such as but not limited to a weight percent ratio of 100:1 to 1:100 of monoadduct to bisadduct. The treatment composition further includes the monoadduct alone, the bisadduct alone and/or these variations of concentrations of monoadduct and bisadduct along with minor (as little as 0.1 wt %) to major amounts (as much as 85 to 98 wt %) of unreacted starting materials relative to the amount(s) of mono and/or bisadduct including but not limited to the corresponding maleic starting material and the corresponding diamine starting material.

Preferred embodiments of the Michael Addition product comprise the monoadduct as the 2-(multi-organo- or siliconyl-amino)succinic acid, ester, amide, or imide optionally in mixture with the bisadduct of the same carbonyl arrangement (e.g., acid, ester, amide or imide). More preferred embodiments comprise the monoadduct acid, and/or ester optionally in mixture with the corresponding bisadduct. Especially preferred is the monoadduct acid or ester, especially the acid. Another preferred embodiment is the bisadduct acid or ester, especially the acid.

When either or both monoadduct and bis adduct are carboxylic acid embodiments, they have the dual ionizable character of carboxylic acid and amine groups. This dual character gives rise to several ionic and neutral forms depending upon the acidity or basicity of the treatment composition. These forms are described below in the pH control section.

The ester, amide and imide embodiments of the monoadduct and/or bisadduct are also ionizable. While they also give rise to one several ionic and neutral forms depending upon the acidity or basicity of the treatment composition, these forms are not as complex as the adducts with carboxylic acid. These forms of the ester, amide and imide embodiments are also described below in the pH control section.

The aqueous, organic, or aqueous-organic medium may be water or an organic alcohol or a mixture of water with an alcohol and each may optionally include a non-alcohol organic solvent.

The treatment composition may optionally comprise one or more of a film forming polymer, a nail plate penetration enhancer, a fatty alcohol, a rheology control agent, a nonionic surfactant, a colorant, and a perfume, preferably at least one of a nail plate penetration enhancer, a rheology control agent and a nonionic surfactant.

Embodiments of the method include one or more applications, preferably multiple applications, to a clean, dry nail plate of a flowable formulation, a flowable-gel formulation or a gel formulation of one of the compositional embodiments at a concentration as described below. An applied coating is allowed to remain in at least semifluid/gel state on the nail plate for a period of 0.5 to 15 minutes, preferably a period of 1 to 10 minutes before drying. The so-coated nail plate may be optionally recoated with the flowable coating after a period of 10 minutes to one hour. The one or more coatings may be air dried with optional application of mild heat.

The invention as well is directed to embodiments of the treatment composition comprising one or more of the monoadduct, the bisadduct, the mixture thereof and their combination with unreacted starting material as described above. The treatment composition contains no aldehyde components which are traditionally found in nail strengthening formulations.

### Definitions

Unless defined otherwise, all technical and scientific terms used herein have the same meaning as commonly understood by a person of ordinary skill in the art.

As used herein and in the appended claims, the singular forms “a,” “an” and “the” include plural referents unless the context clearly dictates otherwise.

The term “about” as used herein, when referring to a numerical value or range, allows for a degree of variability in the value or range, for example, within 10%, or within 5% of a stated value or of a stated limit of a range.

All percent compositions are given as weight-percentages relative to the total weight of the composition, unless otherwise stated.

All average molecular weights of polymers are weight-average molecular weights, unless otherwise specified.

The term “may” in the present context means or “is able to” and is a synonym for the term “can” and “is.” The term “may” as used herein does not mean possibility or chance.

The term “and/or” means both or all of the items together to which these conjunctions refer as well as each of the items alone and separate from the others. When more than two items are referred, the term and/or also means any combination of these multiple items as well as all and each.

The term “substantially free” as the term is used herein means completely or almost completely; for example, a composition that is “substantially free” of a component either has none of the component or contains such a small amount that any relevant functional property of the composition is unaffected by the presence of the small amount of the component in question. A compound that is “substantially pure” has only negligible traces of impurities present.

The term “substantial” means a significant amount such as more than a majority amount. For example, a mixture of compounds A and B in which A is present in a substantial amount means that A is present at a weight percent or number of moles that is greater than the weight percent or number of moles of B. This term also means more than a minimal characteristic, examples of which include substantial flow or substantial treatment.

The following groups of terms are used throughout this application: 1) preferred, preferably and preferable; 2) more preferred, more preferably and more preferable, 3) especially more preferred, especially more preferably and especially more preferable; 4) most preferred, most preferably and most preferably; 5) especially most preferred, especially most preferably and especially most preferable; and 6) very especially most preferred, very especially most preferably and very especially most preferable. These groups convey a meaning of preference for a group of substituents, structures, moieties, components and compounds. The degree of preference is self-explanatory by the terms themselves. Within each group, the meanings of the synonyms, preferred, preferably and preferable are the same. There is no difference in meaning in the context of this application when a group is described in a particular sentence as preferred and then in another sentence this same group is described as preferably. Not all six categories of preference are used in this application to describe each substituent, formula, subgenus integer symbol and atom designator. In some instances, two or three categories are used while in other categories five or six categories are used. The degree of preference as expressed by these terms for members of series which progress from many to a few individually named components is self-explanatory and internally consistent for the particular series being described.

Reference throughout this specification to “embodiment”, “one embodiment” or “preferred embodiment” or “more preferred embodiment” or “most preferred embodiment” means a feature of any one or more elements and can be used in connect with different elements of the invention. A particular feature, structure, or characteristic described in connection with an embodiment may not be the same as another feature, structure or characteristic of another embodiment of the presently claimed invention. Thus, appearances of the phrases “in one embodiment” or “an embodiment” or “in a preferred embodiment” in various places throughout this specification are not necessarily all referring to the same embodiment but may refer to different embodiments of the presently claimed invention. Furthermore, the features, structures, or characteristics in one or more embodiments may be combined in any suitable manner, as would be apparent to a person skilled in the art from this disclosure. Furthermore, while some embodiments described herein include some, but not other features included in other embodiments, combinations of features of different embodiments are meant to be within the scope of the subject matter, and form different embodiments, as would be understood by those skilled in the art. For example, in the appended claims, any of the claimed embodiments can be used in any combination.

The term film former means a fully formed polymer such as a poly(meth)acrylate, polyester, polyamide, polyurethane, cellulosic ether or ester or shellac that will form a contiguous film or layer when the film former in an organic or aqueous medium is coated on a substrate and dried to remove the medium. The film former typically does not undergo substantial cross-linking or other chemical reaction after its deposition as a film.

The term surfactant means a zwitterionic, nonionic, anionic or cationic compound having lipophilic and hydrophilic qualities so that it can function to solubilize lipophilic substances in hydrophilic and/or aqueous media.

The term rheologic control agent means a rheology modifier that will thicken an otherwise free-flowing liquid composition. The modification renders the composition flowable when applied by spray, brush or other coating method but the composition will remain in a static position in a quiescent state. These agents are typically classed as thixotropic agents. Examples include polyvinyl alcohol, a polyethylene glycol, vegetable gum, laponites, and hydrocarbon wax.

The term alcohol means a mono, di, tri or polyol that can solubilize other components of the composition. Alcohol includes methanol, ethanol, propanol, butanol, ethylene glycol (ethylene diol), glycerin (glycerol), propylene diol, or di-ethylene glycol, di-propylene glycol as well as glyme (methoxylated ethylene glycol). Preferred alcohols include but are not limited to methanol, ethanol, propanol, ethylene glycol, propylene glycol and glycerin.

The term penetration agent means an organic compound that facilitates penetration of organic solids through the dermis and/or corneum layers of nail plates. Examples include dimethyl sulfoxide, lauryl sulfate, dimethyl formamide, glycerol and azone.

The term gel means a liquid within a three-dimensional network that forms at least a semi-solid-like consistency in the static, quiescent state. The gel has sufficient rheologic control and/or density at rest (static state) to maintain continuous integrity of the liquid gel as a coating or layer on a flat or curved surface. The gel character of the liquid means that the liquid will not spontaneously flow off a surface on which it has been coated but can be readily removed by mechanical force such as by wiping with a cloth or tissue. According to the present invention, the treatment composition may be a gel when a rheology control agent is included therein.

The term flowable means a liquid that will flow like water or an aqueous latex paint when contacted by mechanical means such as by a brush, sponge or other applicator.

Together, the terms gel and flowable mean that the liquid having these characteristics is thixotropic.

The term alkylenyl used in the present invention means a linear C2-C6 hydrocarbon chain with open valences at both termini. An example is hexylenyl of the formula —(CH₂)₆—.

The term nail plate means a human fingernail, a human toenail.

### DETAILED DESCRIPTION OF THE INVENTION

The present invention provides a method for prevention and/or treatment of a painful, difficult to heal condition of human nail plates. Weak, brittle and/or inflexible nails are susceptible of cracking, breaking, fracturing and/or splitting down to soft tissue nail beds. When this happens, the person suffers pain that at times is unbearable. Treatment according to the present invention alleviates the pain and helps the nail plate heal.

### Components of the Composition

The treatment aspects of the invention are achieved at least in part by application to damaged nail plates of the treatment composition embodiments of the invention.

A first treatment composition embodiment comprises the Michael addition monoadduct which is a 2-(multi-organo or siliconyl-amino)succinic acid, ester, amide, anhydride or imide or any combination thereof.

A second treatment composition embodiment comprises the Michael addition bisadduct which is the N,N′-bis(2-succinoylhydroxy)-, N,N′-bis(2-succinoyloxy alkyl ester)-, N,N′-bis(2-succinamido)-, N,N′-bis(2-succinanhydrido)- or N,N′-bis(2-succinimido)-1,ω-multi-organo or siliconyl-diamine or any combination thereof.

A third treatment composition embodiment comprises a mixture at any weight ratio of the Michael addition monoadduct and the Michael addition bisadduct as indicated by the phrase monoadduct and/or bisadduct.

A fourth treatment composition embodiment comprises one or more of the first, second and/or third embodiments in combination with compounds used in the production of the Michael addition product embodiments including but not limited to starting material residues such as but not limited to unprotonated diamines, diamine salts, unsaturated carboxylate compound salts, amides formed between the diamines and unsaturated carboxylic acid compounds and similar non-Michael addition compounds formed with the diamine and unsaturated carboxylate compound and unreacted starting materials such as the diamine and unsaturated carboxyl compound.

Embodiments of the monoadduct comprise an a-amino multi-organo carboxy compound of Formula VI and/or an α-amino multi-organo compound of Formulas IXA and IXB.

Embodiments of the bisadduct comprise a bis-α-amino multi-carboxy compound of Formula VII or a bis-α-amino multi-organo carboxyl compound of Formulas XIA and XIB.

Embodiments of the siliconyl monoadduct comprise the monoadduct with siloxane chain of Formula XIIIA and the monoadduct with branched siloxane chain of Formula XIIIB.

Embodiments of the siliconyl bisadduct with a siloxane chain comprise the bisadduct with siloxane chain Formula XIVA or the bisadduct with branched siloxane chain Formula XIVB.

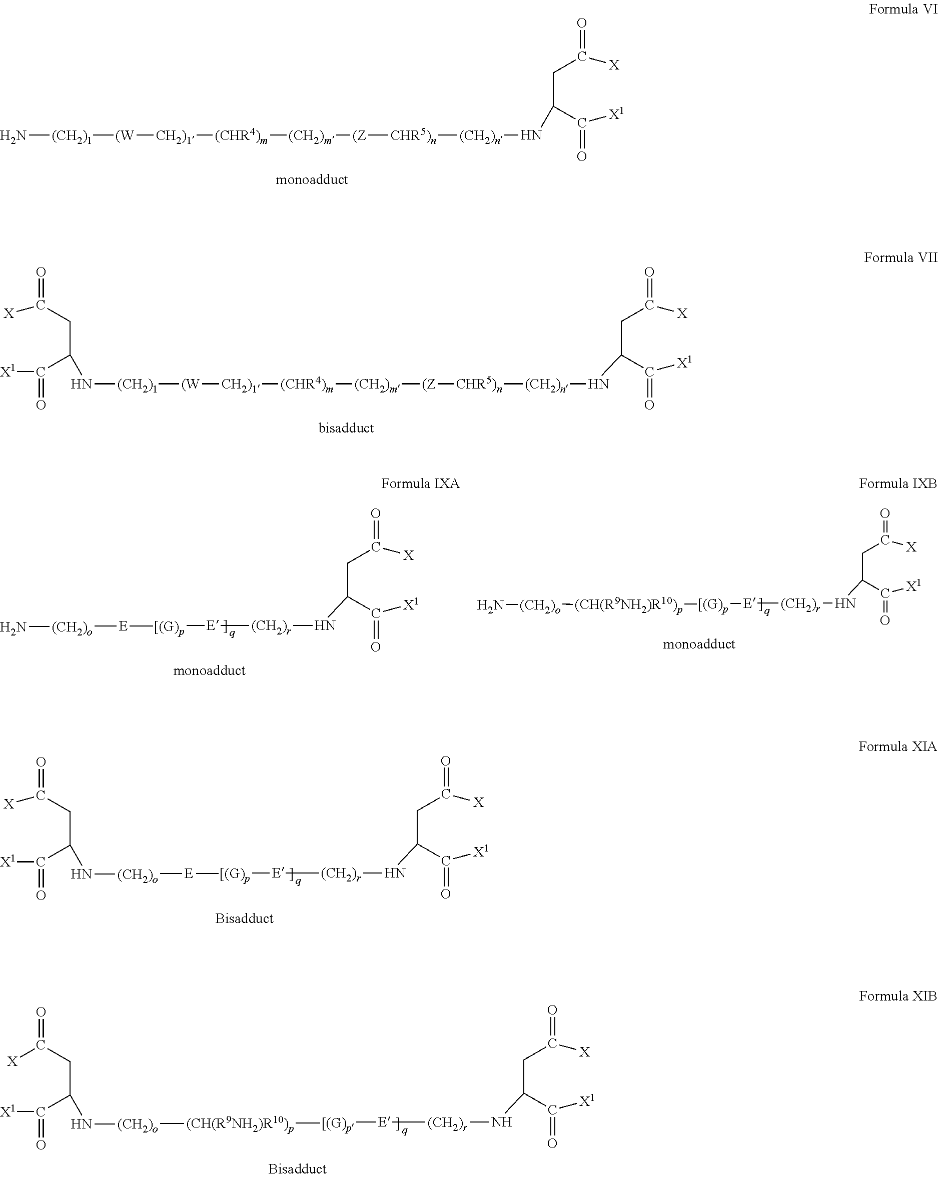

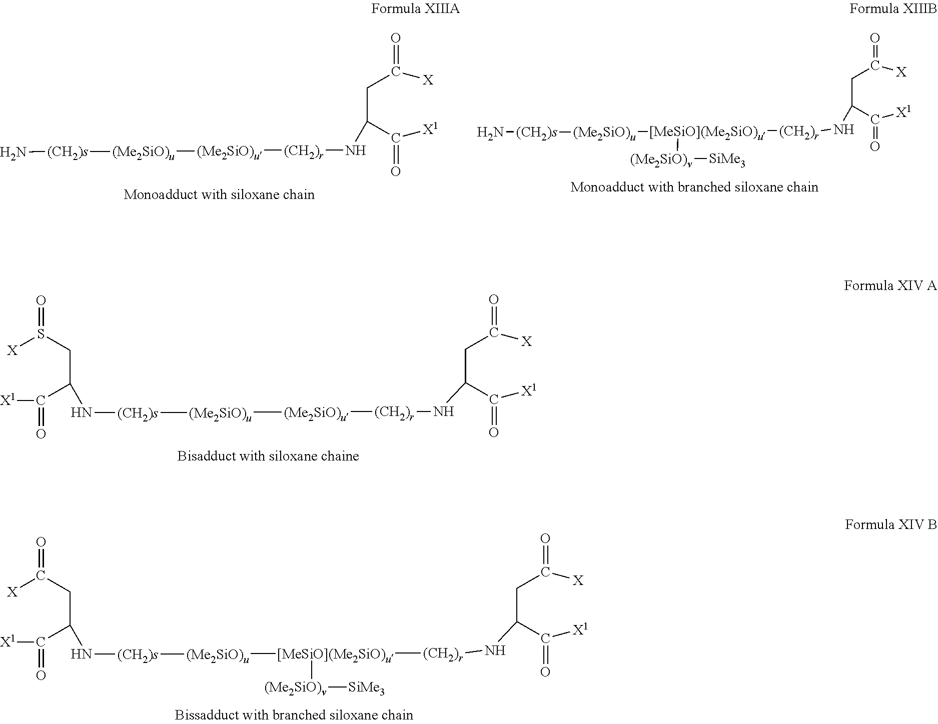

For Formulas VI, VII, IXA, IXB, XIA, XIB, XIIIA, XIIIB, XIVA, XIVB the designators l, l′, m, m′, n, n′, k, o, p, q, r, s, u, u′ and v and substituents X, X′ W Z, E, E′ and G are defined to provide:
1) l, l′, m, m′, n and n′ each independently may be zero or an integer of from 1 to 100. The sum of l, l′, m, m′, n and n′ may be at least 2 and as much as 100, preferably up to 50, more preferably up to 30, even more preferably up to 20 with individual sums being 2, 3, 4, 5, 6, 7, 8, 9, 10, 11, 12, 13, 15 or 15.
2) Designators o and r each independently may be zero or an integer of from 1 to 4.
3) Designator s may be an integer of from 1 to 4.
4) Both of X and X¹ may be —OH, —OR² or —NHR³.
5) W and Z each independently may be O, S or NR⁶.
6) R² is C1-C4 linear alkyl.
7) R³ is H, C1-C4 linear alkyl.
8) R⁴ and R⁵ each individually and separately may be hydrogen or C1-C10 alkyl optionally substituted by one or more of a C1-C4 alkyl group, a C1-C4 alkoxy group, a C1-C4 alkthio group, a C1-C4 alkyl substituted by —CO₂—C1-C4 alkyl group, a C1-C4 alkyl substituted by —CONH—C1-C4 alkyl group, a C1-C4 alkyl substituted by —SO₂—C1-C4 alkyl group, and a C1-C4 alkyl substituted by —SO₂—NH—C1-C4 alkyl group.
9) R⁶ may be hydrogen or a C1-C20 linear or branched alkyl group.
10) Both of X and X¹ may be —OH, —OR² or —NHR³ wherein R² and R³ are defined as given for Formulas VI and VII.
11) E may be cyclopentyl, cyclohexyl, C1-C4 mono, di or trialkyl cyclohexyl, phenyl or C1-C4 alkyl phenyl, C6-C10 aryl with 1 or 2 rings or C1-C4 mono- or multi-alkyl C6-C10 aryl with 1 or 2 rings, pyridinyl, 1,3,5-triazinyl or alkyl- or phenyl-substituted 1,3,5 triazine.
12) E′ may be cyclopentyl or cyclohexyl, C1-C4 mono, di or trialkyl cyclohexyl, phenyl or C1-C4 alkyl phenyl.
13) G may be —(CH₂)ₖ—, —SO₂—, —NH—.
14) k may be an integer of from 1 to 10.
15) p, p′ and q each independently may be zero or 1.
16) u, u′ and v each independently may be zero or an integer of from 1 to 50, preferably 1-20, more preferably 1-10, most preferably 1-5.
17) R⁹ may be hydrogen or —(CH₂)ₖ—.
18) R¹⁰ may be hydrogen or a C1-C6 alkyl.

Alternatively, for Formulas VI, VII, IX and XI, X and X¹ together with their corresponding carbonyl groups may form an anhydride of the formula —OC—O—CO— or an imide of the formula —OC—NR³—CO—.

Preferred embodiments of the monoadduct and/or the bisadduct are the carboxylic acid embodiment and the alkyl ester embodiment. More preferred embodiments are the carboxylic acid embodiment and methyl ester embodiment of the monoadduct and/or bisadduct. An especially more preferred embodiment is the carboxylic acid embodiment of the mono and/or bisadduct. The most preferred embodiment is the carboxylic acid embodiment of the monoadduct as well as its mixture with the carboxylic acid bisadduct in which the weight ratio of monoadduct to bisadduct ranges from 100:1 to 2:1, preferably from 50:1 to 5:1, more preferably from 50:1 to 20:1. Optionally, these preferred, more preferred and most preferred forms of the Michael addition product may but not necessarily include from 0.1 wt % to 95 wt %, preferably from 0.1 wt % to 75 wt %, more preferably from 1 wt % to 50 wt %, especially more preferably from 1 wt % to 30 wt %, most preferably from 1 wt % to 25 wt % of starting material residue(s) and/or unreacted starting material(s) including the starting diamine(s) and unsaturated carboxy compound, relative to the total amount of mono and/or bisadduct and starting material residue(s) and/or unreacted starting material(s). Exemplary concentrations of the starting material residue(s) and/or unreacted starting material(s) may range in 1 wt % increments from the minimum to maximum wt %' s for each of the foregoing ranges, e.g., 1 wt % to 25 wt % in 1 wt % increments.

The concentration of the monoadduct and/or bisadduct with or without starting material residue(s) and/or unreacted starting material(s) in the treatment composition may be at least 0.2 wt percent, and preferably may range from 0.2 wt percent to about 40 wt percent, more preferably from about 0.2 wt percent to about 25 wt percent, most preferably from about 0.2 wt percent to about 20 wt percent and especially most preferably from about 0.2 wt percent to about 6.5 wt % to about 15 wt percent or 0.2 wt percent to about 6.5 wt percent to 10 wt percent. Exemplary concentrations include 1.5 wt %, 2.0 wt %, 2.5 wt %, 3.0 wt %, 3.5 wt %. 4.0 wt %, 4.5 wt %, 5.0 wt %, 5.5 wt %, 6.0 wt %, 6.5 wt %, 7.0 wt %, 7.5 wt %, 8.0 wt %, 8.5 wt %, 9.0 wt %, 9.5 wt %, 10.0 wt % and all 0.1 wt % intervals between these exemplary concentrations. The weight percentages are relative to the total weight of the composition including any optional components.

The above-described embodiments of the monoadduct and/or bisadduct as well as the optional starting material residue(s) and unreacted starting material(s) can have ionized and neutral electronic arrangements depending upon the pH of the medium. These variations produce molecules of these embodiments having forms such as uncharged neutral, zwitterionic, free amine, cationic and anionic forms (together variable charge embodiments). These forms of the monoadduct and/or bisadduct as well as the optional starting material residue(s) and unreacted starting material(s) constitute a variation of mixtures thereof depending upon the pH of the medium. The genesis of these forms is explained in the pH control section.

These forms of the embodiments of the mono adduct and bisadduct as well as the optional starting material residue(s) and unreacted starting material(s) include:
a) An embodiment with at least a portion of the monoadduct and/or bisadduct as well as the optional starting material residue(s) and unreacted starting material(s) with carboxylic acid in which the carboxylic acid and amine are un-ionized and unprotonated respectively so as to provide at least in part an uncharged neutral embodiment;
b) An embodiment with at least a portion of the monoadduct and/or bisadduct as well as the optional starting material residue(s) and unreacted starting material(s) with carboxylic acid in which the carboxylic acid protonates the amine so that the anion of the carboxylate group and cation of the ammonium group are balanced to provide at least in part a zwitterionic embodiment carrying no net charge;
c) An embodiment with at least a portion of the monoadduct and/or bisadduct with ester, amide or imide groups as well as the optional starting material residue(s) and unreacted starting material(s) having un-neutralized free amine groups so as to provide at least in part a free amine embodiment;
d) An embodiment with at least a portion of the monoadduct and/or bisadduct as well as the optional starting material residue(s) and unreacted starting material(s) with carboxylic acid neutralized as an anionic group so as to provide at least in part an anionic embodiment; and
e) An embodiment with at least a portion of the monoadduct and/or bisadduct as well as the optional starting material residue(s) and unreacted starting material(s) with carboxylic acid, ester, amide or imide groups having their amine group(s) neutralized as a cationic group(s) so as to provide at least in part a cationic embodiment.

These electronic forms (i.e., the uncharged neutral, zwitterionic, free amine, anionic and cationic embodiments) typically are multiply present in the total amount of monoadduct and/or bisadduct present. The multiple forms present may be rebalanced to change from one form to another, or to adjust the portions of several of the forms present by adjustment of the pH of the medium of the treatment composition. As explained in the pH control section, these forms constitute pH managed portions of the monoadduct and/or bisadduct as well as the optional starting material residue(s) and unreacted starting material(s). The total amount of monoadduct and/or bisadduct as well as the optional starting material residue(s) and unreacted starting material(s) may constitute several of these portions such that one form will be present along with other complimentary forms and the amounts and identities of the portions of these forms can vary depending upon the pH control provided by the medium.

### Preparation of Monoadduct and/or Bisadduct

The mono- and/or bis-adduct carboxylic acid embodiments may be prepared by Michael addition synthesis with the starting materials including the multi-organo diamines of Formulas V, VIIIA, VIIIB, the siliconyl diamines of Formulas XIIA, XIIB and the unsaturated carboxylic compound (such as a maleic compound) of Formula IV:

H₂N—(CH₂)ₗ—(W—CH₂)_{l′}—(CHR⁴)ₘ—(CH₂)_{m′}—(Z—CHR⁵)ₙ—(CH₂)_{n″}—NH₂ Formula V

H₂N—(CH₂)ₒ-E-[(G)ₚ-E′-]_{q}-(CH₂)ᵣ—NH₂ Formula VIIIA

H₂N—(CH₂)ₒ—(CH(R⁹NH₂)R¹⁰)ₚ-[(G)_{p′}-E′-]_{q}-(CH₂)ᵣ—NH₂ Formula VIIIB

H₂N—(CH₂)ₛ-(Me₂SiO)ᵤ-(Me₂SiO)_{u′}—(CH₂)ᵣ—NH₂ Formula XIIA

H₂N—(CH₂)ₛ-(Me₂SiO)ᵤ-[MeSiO(SiMe₂O)ᵥ—SiMe₃)](Me₂SiO)_{u′}—(CH₂)ᵣ—NH₂ Formula XIIB

X—OC—HC═CH—CO—X Formula IV

The designators l, l′, m, m′, n, n′ k, o, p, p′, q, r, s u, u′, v and r and substituents W, Z, R⁴, R⁵ E, E′ and G of Formula V are as given above for Formulas VI and VII IXA, IXB, XIA, XIB, XIIIA, XIIIB, XIVA and XIVB.

The substituents of Formula IV designated as X may both be —OH, —OR² or —NHR³. The substituent R² is C1-C4 linear alkyl. The substituent R³ is hydrogen or is a C1-C4 linear alkyl. Together these definitions of X provide the maleic compound of Formula IV as a maleic acid, a dialkyl maleate with both alkyls being a C1 to C4 linear alkyl group, a maleamide (R³ being H) or an N,N′-dialkyl maleamide with both alkyls being a C1 to C4 linear alkyl group.

Alternatively, the maleic compound of Formula IV may have the substituents X and X together with their CO groups formed into an anhydride of the formula —C═O—O—C═O—, or an imide of the formula —C═O—NR³—C═O— with R³ being the same as designated for the amide. This alternative provides Formula IV as maleic anhydride or as maleimide which is optionally N substituted with a C1 to C4 alkyl group.

As a starting material, Formula IV with has the substituents X the same in both positions. Both are hydroxyl, alkoxy or alkamido. When Formula IV reacts with the diamine to form the addition product Formula I and/or Formula II, the X of the X—OC— adjacent to the unsaturated carbon residue to which the amine nitrogen is bonded becomes X¹.

Exemplary multi-organo- and siliconyl-diamine classes include but are not limited to linear aliphatic diamines, cycloaliphatic diamines, aromatic diamines, polyether diamines, polyimines, polyamide diamines, amido diamines, silicone diamines and diamino silanes. Preferably, these exemplary classes of diamines are soluble in ethanol or water, have a preferred molecular weight of from about 100 Da to about 5000 Da, preferable up to about 500 Da, have a melting point of lower than about 42° C., more preferably are liquid at STP and the amine groups forming the reactive diamine moiety are primary amine groups.

Species of these exemplary classes of diamines include but are not limited to diethylenetriamine, dipropylene triamine, tetraethylen pentaamine, laurylaminedipropylene diamine, trimethylhexamethylene diamine, propyleneoxide triamine such as Baxxodur EC310 or EC311 manufactured by BASF, methylene dianiline, m-xylylene diamine, p-phenylene diamine, o-phenylene diamine, diaminophenyl sulfone, 4,4′diaminobiphenyl, benzoguanamine, isophorone diamine such as Vestaminpacm manufactured by Evonik or Epikure3300 manufactured by Momentive, methyldiaminocyclohexane, 4,4′diaminodicyclohexylmethane, diaminocyclohexane, 3,3′dimethyl-4,4′-diaminocyclohexylmethane, 1,8-diamino-p-menthane, 1,3-bis(aminomethyl)cyclohexane, serotonin, polyoxypropylenediamine such as Jeffamine D-230, D400 T403 and similar Jeffamines manufactured by Huntsman or Baxxodur EC302 or CE301 manufactured by BASF, 4,9-dioxadodecane-1,12-diamine, poly(propylene oxide triamine, versamid amidoamine by Gabriel performance produces, Genamid 490 amidoamine by BASF, bis-aminopropyl dimethicone, aminopropyl dimethicone, Amodimethicone, N-(2-aminoethyl)-3-aminoproyl methyldimethoxysilane, N-(2-aminoethyl)-3-aminopropyltrimethoxysilane.

When the Michael addition synthesis involves maleic anhydride and the reaction conditions are aqueous and/or there is acid or base workup, the anhydride group undergoes ring opening to form the carboxylic acid groups. While the anhydride embodiments may be synthesized from the maleic anhydride in aprotic organic medium and isolated by non-aqueous chromatography, subsequent preparation of the treatment composition with the aqueous or aqueous-organic medium and an adjustment of pH will inevitably hydrolyze the anhydride. For these reasons, the embodiments of the anhydride and the carboxylic acid may be regarded as at least substantially equivalent in use.

The ester, amide and imide embodiments of the mono- and/or bisadduct may be prepared by Michael addition synthesis with the corresponding starting materials, alkane diamine and the maleic alkyl ester, N,N′-bis-(R³) maleamide or the NR³-maleimide as shown by Formula IV. Alternatively, the ester, amide and imide embodiments of the monoadduct and/or bisadduct may be made by converting the monoadduct and/or bisadduct with X as carboxylic acid to the corresponding ester, amide or imide embodiments according to carboxylic acid conversions described for example in J March, Advanced Organic Chemistry, 4^{th} Ed. Wiley Interscience Publication, Jon Wiley & Sons New York, 1992.

The molar ratios for the diamine and maleic starting materials trace the molar ratios between the residues of these two starting materials as the adducts of Formulas VI, VII, IXA, IXB, XIA, XIB, XIIIA, XIIIB, XIVA, XIVB. If the monoadduct is to be produced, the molar ratio of the diamine of Formula V, VIIIA, VIIIB, XIIA or XIIB to the maleic compound Formula IV is at least 2 and preferably greater than 2, including 1/10 molar increments from a molar ratio of 2 and higher to 3 or 4. If the bisadduct is to be produced, the molar ratio of the maleic compound, Formula IV to the diamine of Formulas V, VIIIA, VIIIB, XIIA or XIIB is at least 2 and preferably greater than 2 (reverse of the molar ratio for producing the monoadduct). The molar ratio is calculated by determining how many times a single diamine of Formula V, VIIIA, VIIIB, XIIA or XIIB and VIII is to react with the maleic starting material Formula IV. If the bis adduct is to be produced, two maleic molecules, Formula IV, react with one diamine molecule, Formula V, VIIIA, VIIIB, XIIA or XIIB due to the presence of two primary amine groups for each diamine molecule and a single conjugated ethylenic group for each maleic molecule. If the monoadduct is to be produced, one diamine molecule is reacted once with one maleic molecule. Because the diamine molecule has a second primary amine, these molar ratios assure that predominant combination produced is the monoadduct or the bis adduct. As with any addition reaction, both possibilities are possible, however, the predominance of the mono adduct versus the bis adduct is greatly managed by the variation of the molar ratio of starting materials. Use of some excess of diamine above the 2 molar ratio for production of the monoadduct and use of some excess of maleic compound above the 2 molar ratio for production of the bis adduct heightens the predominance of the mono adduct or bis adduct respectively. In each instance excess unreacted starting material may be removed during the work-up of the reaction mixture or may be allowed to remain as part of the composition for treatment.

Experimental procedures for preparation of the mono and bis adducts may follow those outlined in U.S. Pat. Nos. 3,158,635; 5,846,925 and 6,465,690, the disclosures of which are incorporated herein by reference.

### The Medium

The liquid medium of the composition for treatment is an aqueous, organic or aqueous-organic liquid mixture. The organic medium or the organic solvent of the aqueous-organic medium may be an alcohol alone or in combination with a non-alcohol selected from a ketone, ester, acetate, dimethyl isosorbide, DMSO, DMF, THF or any mixture or combination thereof.

The alcohol may be a mono or polyalcohol or a mixture of such alcohols. Preferably, the alcohol component is a monoalcohol such as methanol, ethanol or propanol or a combination of one or more monoalcohol with polyols such as glycerol or ethylene glycol or propylene glycol or a combination thereof. Any mixture of any combination of the mono and polyalcohols may be employed.

The non-alcohol selected from ketone, ester, acetate, dimethyl isosorbide, DMSO, DMF and THF is water and alcohol soluble. The ketone may be acetone or methyl ethyl ketone or a mixture thereof. The non-alcohol can be combined with one or more alcohols described above to form the organic medium or the organic portion of the aqueous-organic medium. The volume: volume ratio of non-alcohol to alcohol may be about 1:100 to 1:10, preferably 1:50 to 1:20. Preferably, no non-alcohol is included with the organic medium and with the organic portion of the aqueous-organic medium.

The concentration of the organic liquid component in the aqueous-organic medium may range from about 1 wt percent to about 100 wt percent or a volume: volume ratio of organic liquid to water of from 1:50 to 2:1, preferably from about 1:50 to about 1:2. The remainder is water for the aqueous-organic medium and is all water for the aqueous medium.

### The pH Control of the Treatment Composition

The control of the pH of treatment composition manages at least in part the greater or lesser ability of the monoadduct and/or bisadduct to penetrate or permeate into the interior portion of the nail plate and strengthen the nail plate. Some penetration of the nail plate may be accomplished with all embodiments of the monoadduct and/or bisadduct at pH's ranging from highly acidic such as about 1.5 or 2 to highly basic such as about 10 or 11.5. Preferable pH's range from about 2 to about 11, more preferably from about 2.5 to about 10.5. The greater or lesser ability to penetrate within these pH ranges depends at least in part upon the acidic, basic or zwitterionic nature of the monoadduct and/or bisadduct.

Permeation of organic compounds into the nail plate has been examined extensively. See for example S. Murdan et. al., Skin Pharmacol. Physiol., 2011;24(4):175-81; S Baswan, et. al. Mycoses 2017 May; 60(5) 285-295 Pub Med Central PMC5383514 and D. Mertin et al, J. Pharm. Pharmocol., 1997, 49, 30-34 (1996).

These authors explain that nail plate permeation of organic compounds is dependent at least in part upon the hydrophobicity/hydrophilicity and also upon the pH of medium containing the organic compounds when they are acidic or basic. Although at least some permeation occurs throughout a very wide pH range for acidic and basic organic compounds, management of the pH to provide a greater number of molecules of the compound having a non-ionic, undissociated character enables more effective transport into the nail plate. see S. Baswan cited supra, page 7.

For example, Mertin discloses that benzoic acid produces a permeability coefficient of 78.6 at pH 2 but its permeability coefficient drops to 8.29 at pH 7.4. This variable effect of pH can be understood when the pKa of benzoic acid is considered. The pKa is the dissociation constant when half of the benzoic acid is dissociated (i.e., benzoate anion) and half is undissociated (i.e., benzoic acid). The pH at this point is the same and for benzoic acid that pH is 4.2 so that at this pH, only half of the benzoic acid is non-ionic and undissociated. When the pH is dropped to 2 as Mertin did, a benzoic acid is obtained that is almost completely undissociated. In other words almost all of the benzoic acid molecules at this pH are electrically neutral. Hence, these authors show that while benzoic acid can penetrate a nail plate throughout a pH range from very acidic to basic, the amount of benzoic acid penetrating the nail plate relative to the total amount dissolved in the carrier medium (water) is the portion that is undissociated. This undissociated portion depends upon the pH of the medium and the pKa of the compound. Consequently, these studies of the nail plate permeability coefficient for an organic acid show that permeability increases as the portion of undissociated organic acid becomes greater. This relationship is dependent upon the pH of the medium for the organic acid and the pKa of the organic acid. The same applies to organic bases.

These scientific studies illustrate that while nail plate penetration for an acidic or basic organic molecule can occur throughout a wide pH range, the number of molecules able to penetrate depends upon control of the pH of the medium containing the acidic or basic organic compound. Control of the pH relative to the acidic or basic character of the organic compound enables a higher or lower number of the organic compound molecules to remain in an undissociated form and penetrate into the nail plate.

According to these scientific studies, the pH of the treatment composition medium according to the invention affects the number of monoadduct and/or bisadduct molecules as well as the optional starting material residue(s) and unreacted starting material(s) penetrating the nail plate and bringing about nail strengthening. While a pH range from highly acidic to highly basic will enable at least some penetration of these monoadduct and/or bis adduct molecules, there will be a “sweet spot pH” which will maximize the number of monoadduct and/or bisadduct molecules as well as the optional starting material residue(s) and unreacted starting material(s) able to penetrate the nail plate.

The corresponding pH behavior of the mono and/or bisadduct with carboxylic acid differs from that of benzoic acid or an organic amine because the adducts have both carboxylic acid groups and amine groups. Like amino acids, the adducts are zwitterionic so that they have a balance between the carboxylic acid and amine groups. In a medium at low pH, the carboxylic acid group of the adducts will be almost completely undissociated or un-ionized but at this low pH, the amine group(s) will be almost completely protonated. The reverse begins to appear as the pH is raised so that at high pH carboxylate anions and free amine groups will primarily be present. Accordingly, the change of pH will manage the distribution of molecules of monoadduct and/or bisadduct having differing combinations of carboxylic acid groups, carboxylate groups, protonated amine groups and free amine groups. There will be a particular pH region between highly acidic and highly basic where the pH will produce a distribution of adduct molecules providing the highest number of undissociated molecules. This is the “sweet spot” pH range where this number of adduct molecules with un-ionized carboxylic acid groups and free amine groups will be highest. In other words, as a function of pH, the portion of these molecules in undissociated form follows a gaussian curve. This gaussian curve also shows the degree of consequent nail strengthening by the monoadduct and/or bisadduct embodiments with carboxylic acid groups as a function of pH. At least some penetration and strengthening will be produced throughout this pH range but penetration of the highest number of undissociated molecules and a strongest nail strengthening is achieved by this “sweet spot” pH range.

In general, these studies show that a medium with a pH ranging from a highly acidic pH of 1 or 2 to a neutral pH of 6.5 to 7.5 to a highly basic pH of 10.5 to 11, preferably from about 2 to about 10, more preferably about 2 to about 7, especially more preferably about 2.5 to about 5 will be suitable for providing a strengthening effect for a treatment composition containing the monoadduct and/or bisadduct embodiments with carboxylic acid. These studies also show that the sweet spot for the mono and/or bis adduct embodiments with carboxylic acid groups may be estimated by the isoelectric points of the amino acids aspartic acid and glutamic acid. Similar to the monoadduct and/or bisadduct, these amino acids have two carboxylic acid groups and one amine group. The isoelectric points for these amino acids average at about pH 3. Hence, while a highly acidic to highly basic pH range for the treatment composition is suitable for nail strengthening of the monoadduct and/or bisadduct with carboxylic acid, the “sweet spot” pH range will be about 2 to about 5, preferably about 2.5 to 3.5.

The “sweet spot” for the ester, amide, anhydride and imide embodiments of the monoadduct and/or bisadduct (hereinafter amine monoadduct and/or bisadduct embodiments) as well as the optional starting material residue(s) and unreacted starting material(s) differs from the that of the mono and/or bisadduct embodiments with carboxylic acid groups. Because they only have protonatable amine groups, their “sweet spot” is determined by the basicity of the amine group. While the amine monoadduct and/or bisadduct embodiments also show nail strengthening over a wide pH range, a strongest nail strengthening will be this “sweet spot.” The “sweet spot” is at least in part determined by a basic pH providing the unprotonated forms of these amine adducts, i.e., the free amine embodiments. Mertin found that although at least some permeation for pyridine occurred at pH 2, a high permeation coefficient for pyridine was obtained at pH 7.4. When the conjugated acid pKa of pyridine (5.25) is considered, Mertin's basic pH finding indicates that a pH “sweet spot” for amines will be higher than the amine's conjugate acid pKa.

Because the amine monoadduct and/or bisadduct embodiments have probable conjugate acid pKa's at least somewhat higher the conjugate acid pKa of pyridine (5.25), their pH sweet spots may also be higher than 5.25. A very basic pH such as 11.5 or higher, however, can cause cleavage of the ester, amide or imide groups and damage the nail plate. See B. Chiego, et. al., J. Investigatory Dermatology, 5(2) 95-103 (1942).

Accordingly, at least some permeability and consequently at least some nail strengthening by the amine adduct and/or bisadduct as well as the optional starting material residue(s) and unreacted starting material(s) may be obtained throughout a pH range of the medium from acidic to basic. Quantitatively this pH range may be from about 2 to about 11. The “sweet spot” pH for the treatment composition of free amine monoadduct and/or bisadduct embodiments as well as the optional starting material residue(s) and unreacted starting material(s) providing a stronger nail strengthening effect may be a range of about 6 to about 10.5, preferably about 7.5 to about 10, more preferably about 7.8 to about 9.5.

### Agents for pH Control

For the electronic forms of the monoadduct and/or bisadduct with carboxylic acid groups, the pH adjustment may be made with addition of acid to the medium of the treatment composition. Appropriate acids include hydrochloric acid, sulfuric acid, acetic acid and/or sodium or potassium bisulfate as well as combinations with acidic buffer systems such as citrate and/or maleate buffer.

For the electronic forms of the monoadduct and/or bisadduct having carboxylate ester, imide or amide groups as well as the optional starting material residue(s) and unreacted starting material(s), the pH adjustment may be made with addition of base to the medium of the treatment composition. Appropriate bases include sodium or potassium hydroxide, bicarbonate or carbonate, borate, ammonia, organic amine and/or buffered systems thereof.

### Optional Constituents of the Treatment Composition

Optional constituents of the treatment composition include one or more of a film former, a nonionic surfactant, a fatty alcohol, a rheologic control agent, a penetrating agent, a perfume, a preservative and alternate diamines. Several combinations of these optional components may be included in the composition but those optional components that may complex, occlude, absorb, adsorb, form electrostatic complexes with and/or otherwise entangle or inhibit or retard the ability of the monoadduct and/or bisadduct to penetrate the nail plate and/or significantly change the pH of the medium should be minimized and/or avoided.

The film former of the composition is a fully formed non-reactive polymer. The film former may be a poly(meth)acrylate, a polyester, a polyamide, a polyurethane, a cellulosic ether or ester or a shellac that will form a contiguous film or layer when the film former in an organic, organo-aqueous and/or aqueous medium is coated on a substrate and dried to remove the medium. The polyester may be a polymer of a hydroxycarboxylic acid or a polymer of a diol and dicarboxylic acid. The polyamide may be a polymer of an amino carboxylic acid or a diamine and a diacid. The polyurethane may be a polymer of a diol and a diisocyanate.

Exemplary film formers include poly methyl or ethyl (meth) acrylate, a copolymer of methyl or ethyl (meth)acrylate and styrene or vinyl methoxide, a styrene/acrylate/ammonium acrylate copolymer, a vinyl copolymer such as polyvinyl butyral or polyvinyl chloride, polylactide, polyglycolide, a copolymer of lactide and glycolide, polyester of C4-C6 alkylene diol and C4-C6 alkylenyl dicarboxylic acid, adipic acid/neopentyl glycol/trimellitic anhydride copolymer, polyamide of C4-C6 alkylene diamine and C4-C6 alkylenyl dicarboxylic acid, a polyurethane of C4-C6 alkylenyl diol and C4-C6 alkylenyl diisocyanate or 1,4-diisocyanatobenzene, methyl, ethyl, and/or propyl cellulose, cellulose acetate, cellulose propionate, nitrocellulose, cellulose acetate butyrate, cellulose butyrate, hydroxyethyl cellulose, hydroxypropyl cellulose, carboxymethyl cellulose, hydroxypropyl methyl cellulose and similar cellulose esters and ethers. These film former polymers do not contain side chains or terminal groups that would render the polymer susceptible of cross linking by actinic (UV) irradiation.

The film former polymers form polymer chain networks and entanglements as they congeal and/or solidify into solid contiguous coatings on fingernails. This three-dimensional physical configuration begins while the film former polymers are in solution or dispersion in the medium. Because of this, the film former polymers can retard and/or inhibit delivery of the monoadduct and/or bisadduct to and through the surface of the nail plate. The polarity and lipophilicity of the film former polymers also may tend to affect the nail plate permeation coefficient of the mono and/or bisadduct. After the film former polymers have congealed and/or solidified, the resulting coating may be resistant to penetration of further treatment composition to the surface of the nail plate. Consequently, it is preferred to begin treatment of fingernails with a treatment composition that does not contain a film former polymer. Multiple treatments of the treatment composition without a film former polymer is preferred. A final treatment with a treatment composition containing a film former polymer may be preferable because the resulting lacquer and/or enamel coating can seal the nail plate surface thereby avoiding or limiting inadvertent removal of the monoadduct and/or bisadduct from the nail plate surface.

The concentration of film former optionally included with the components of the treatment composition according to the invention may range from about 0.1 wt % to about 20 wt %, preferably about 0.1wt % to about 10 wt %, more preferably about 0.2 wt % to about 5 to 8 wt %, especially more preferably about 0.2 wt % to about 3 or 4 wt %.

Surfactants are surface-active agents that can reduce the surface tension of water and enable water insoluble components to be included in the aqueous or aqueous-organic medium. While the term surfactant may include non-ionic, amphoteric, anionic, and cationic surfactants, the surfactant most appropriate for incorporation with the treatment composition comprises a non-ionic surfactant. The charged surfactants such as anionic, cationic and amphoteric surfactants may affect the permeation coefficient of the mono and/or adduct.

More than one nonionic surfactant may be included in the formulation. Examples of nonionic surfactants include sucrose acetate/isobutyrate, ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, polyoxyethylene hydrogenated tallow amide, a polyoxyether of lauryl alcohol or ceteareth-20 and/or combinations thereof.

The nonionic surfactant may be incorporated in the treatment composition at weight percentages relative to the total weight of the composition of from about 0.1 wt % to about 10 wt %, preferably about 0.1 wt % to about 5 wt %, more preferably from about 0.1 wt % to about 2 wt %.

The rheologic agent controls the viscosity of the composition so that preferably the composition is flowable under pressure and is gel like in a steady state (rest), i.e., the rheologic agent is also thixotropic. Preferably, rheologic control provides thixotropic properties to the composition so that it will flow under force of spraying, brushing or otherwise applying but in a static state, the composition is viscous enough to prevent flow.

A rheologic agent useful for the present composition is a polyol of moderate to molecular weight. Polyglycols, polyethers, polyols, polyamides, polyurethanes, polyesters and block copolymers of these polymers have moderate to high intrinsic viscosities and will contribute the rheologic control for accomplishment of the above described flowable/gel properties of the present composition. Exemplary components include polyethylene glycol and polypropylene glycol of about 10 to about 1000 or higher units, polyethers of butylene oxide and/or pentylene oxide having about 20 to more than 1000 or higher units, polyesters such as polylactide, polyglycolide and copolymers thereof, polyurethanes formed from C4-C6 alkyl diisocyanate and C4-C6 alkyl diol as well as block copolymers of such polyesters or polyurethanes with polyethylene glycol and/or polypropylene glycol also serve as useful rheologic control agents of the present composition. These agents may have average molecular weights ranging from 200 Da to about 10KDa, preferably 300 Da to about 5 KDa, more preferably about 400 Da to about 2 KDa.

The concentration of rheologic agent may range from about 0.01 wt % to about 3 wt %, preferably from about 0.01 wt % to about 2 wt %, more preferably from about 0.01 wt % to about 1 wt % and especially about up to 0.5, or 0.2 or 0.1 wt %.

Clays, silicas, starches, gums and their derivatives can act as thixotropic agents but preferably they may be modified to avoid and/or minimize absorption or occlusion of the monoadduct and/or bisadduct. Accordingly, clays, starches, silicas, gums and their derivatives modified with organic material such as may be provided by laponites may be useful to provide the treatment aspect of the composition.

The fatty alcohol serves as an emulsifying agent and/or an anti-agglomeration and/or smoothing agent to promote substantially uniform flow of the composition as it is applied to a nail. The fatty alcohol may be a C8 to a C36 linear or slightly branched alkyl monoalcohol, preferably a C16 to C 20 fatty alcohol or may alternatively be a fatty alcohol of this configuration covalently bound as an ether to a polyethylene glycol or polypropylene glycol having 5 to 20 glycol groups. The concentration of the fatty alcohol may range from 0.01 wt % to about 2 wt %, preferably from about 0.01 wt % to about 1 wt %, more preferably from about 0.01 wt % to about 0.5 wt % and especially about up to 0.3, or 0.2 or 0.1 wt %.

The penetrating agent facilitates transport or carry of other agents through the plate surface into the interior of the plate. These agents are known and include dimethyl sulfoxide (DMSO), dimethyl formamide (DMF), lauryl sulfate, glycerin, dodecylazacycloheptan-2-one (azone), N-methyl pyrrolidone, di and tetra ethylene glycol, lauric, myristic or capric acid, and terpenoids such as citral, menthol, camphor and hydroxyl limonene and mixtures thereof. Additionally, compounds such as di and tri esters of low to moderate molecular weight of from 200 Da to about 1KDa, preferably up to 500 Da such as trimethyl pentanyl diisobutyrate or acetyl tributylcitrate or acetyl tripropyl ascorbate or dihexyl succinate may enable penetration of the mono and/or bisadduct. The lipophilic and quasi polar nature of these esters may contribute plasticization to the nail plate which in turn facilitates penetration and permeation of the mono and/or adduct. The concentration of the penetration agent may range from about 0.001 wt % to about 2 wt %, preferably from about 0.005 wt % to about 1 wt %, more preferably from about 0.01 wt % to about 0.1 wt %.

Perfumes such as cinnamon, cascarilla, safrole, rose, jasmine, mimosa, narcissus, cassie, citrus, apple, strawberry, cherry, lavender, patchouli, sage, violet, rosemary, myrrh, benzoin, pine, fir amber, copal, and related odoriferous esters and odoriferous terpenes are useful as optional components in the present composition. The concentration of the perfume may range from about 0.01 weight percent to about 1 weight percent relative to the total weight of the composition, preferably about 0.01 weight percent to about 0.5 weight percent, more preferably about 0.01 weight percent to about 0.1 weight percent.

Preservatives such as alkyl parabens, phenoxyethanol, benzoic acid, germaben, DMDM hydantoin, diazolidinyl urea are useful as optional components in the present composition. The preservative may range from about 0.001 weight percent to about 1 weight percent relative to the total weight of the composition, preferably about 0.001 weight percent to about 0.05 weight percent, more preferably about 0.001 weight percent to about 0.01 weight percent.

Coloring agents including pigments soluble dyes and metal oxides are also useful as optional components in the present composition.

While any combination of the optional constituents may be included with the treatment compositions according to the invention, inclusion of embodiments of these optional constituents that may complex, occlude, absorb, adsorb, or otherwise entangle the monoadduct and/or bisadduct so as to inhibit, retard or alter the ability of the monoadduct and/or bisadduct to penetrate and permeate the nail plate should be minimized and/or avoided. Optional constituents that may enable increased nail plate penetration, such as the penetrating agents are preferred optional constituents. Minimal to low to moderate concentrations of nonionic surfactant also are preferred optional constituents due to their ability to promote homogeneous single-phase compositions and their ability to render the nail plate more accessible to the permeation and penetration of the mono and/or bisadduct.

### Method of Application

The treatment composition of the invention includes an aqueous or aqueous-organic medium which provides a liquid state of the composition that is free-flowing at least under pressure. The medium in combination with a rheologic control agent optionally delivers a state of the composition that may be free-flowing under all circumstances, may be thick and viscous under all circumstances, such as a gel, or may be free-flowing under pressure such as by brushing but may be thick and viscous at rest, i.e., thixotropic.

For embodiments of the composition that are free-flowing and do not include a rheology control agent, the nail plate may be immersed in the free-flowing liquid composition to deliver the composition to the nail plate. In this embodiment, the nail plate preferably remains immersed in the liquid composition for a sufficient time to allow penetration of the monoadduct and/or bisadduct into the nail plate.

To promote the healing properties of the composition when a nail plate cannot be immersed in the composition, a coating of the composition on the nail plate is kept in place as a gel through inclusion of a rheologic control agent. The rheologic control agent maintains suitable coating density on a substrate so that at least the monoadduct and/or bisadduct of the coating in a viscous static state can effectively penetrate the surface of the nail plate. When the rheologic control agent enables at least in part a gel character for the composition, the viscosity of the composition is preferably managed so that the composition as a gel can spread to a minor degree upon its application onto a nail plate such as by brushing.

The rheologic control of the treatment composition lessens or prevents spontaneous flow of the composition once the composition is coated onto the nail plate. While the rheologic control enables the composition to form a contiguous flowable coating over the nail plate when applied, the rheologic control also enables the coating to maintain its integrity and remain in place once the coating has covered the nail plate. This characteristic allows the coating to maintain contact with the nail plate for a sufficient time to enable penetration of the complex into the internal region of the nail plate.

The treatment composition of the invention is allowed to dry slowly on the nail so that the monoadduct and/or the bisadduct can penetrate the surface of the plate into the internal region of the plate. Penetration allows contact of the mono and/or bis adduct with broken and/or torn parts of the nail plate below the surface of the plate. It is believed that the contact at least in part mends broken peptide bonds of the internal regions of the plate and the underlying nail bed thereby treating the damaged and/or broken nail.

Exemplary methods of the present invention involve application of the formulated composition to the nail for a sufficient time to enable penetration of the monoadduct and/or bisadduct through the plate surface and into the interior of the plate. Penetration also preferably delivers the monoadduct and/or bisadduct to the interface between the nail plate. Preferably, multiple applications of the treatment composition to the nail may be accomplished with from 2 to 20, preferably 3 to 10 multiple applications more preferably at least 4 applications being made with optional drying periods between the applications.

While a combination of the medium and monoadduct and/or bisadduct alone can enable this delivery to the interior of the plate and to the interface, the delivery may be facilitated by the penetration agent, by the nonionic surfactant and by the fatty alcohol. Any or all or any combination of these components can be employed to promote delivery.

The embodiments of the method include immersion and/or coating as described above. In the immersion method, the nail plate is immersed in a free-flowing treatment composition for a sufficient time to enable delivery of the monoadduct and/or bisadduct to the interior of the plate and to the interface.

In the coating method, the nail plate is coated with the treatment composition by painting, brushing sponging, spraying or otherwise applying the coating to the plate. The treatment composition in this instance is preferably formulated to be a flowable gel so that it will substantially uniformly produce a contiguous, complete coating on the plate when applied but will be statically stationary following application so that it will not flow off the plate.

For immersion and for coating, the treatment composition wet with the medium is allowed to remain in contact with the plate for a time sufficient to permit penetration of the complex into the interior of the plate and to preferably contact the nail. Typical times for wet contact range from 2 minutes to multiple hours.

After a sufficient time for contact and penetration is accomplished, the “wet” composition can be dried by air dry techniques optionally using moderate forced air such as from a hair drier or heated air gun. If a film former is present, the drying activity will establish a film or dry coating of the film former on the surface of the nail.

Penetration of the monoadduct and/or bisadduct as well as the optional starting material residue(s) and unreacted starting material(s) is primarily achieved during the “wet” phase of the application of the treatment composition. However, the subsequent “dry” phase of the treatment composition also enables monoadduct and/or bisadduct as well as the optional starting material residue(s) and unreacted starting material(s) to penetrate at least to some extent and especially if one or more of a fatty alcohol, a penetration agent and/or a surfactant are present. Inclusion of a film former that develops a varnish or enamel coating on the nail after the drying step may at least in part slow “dry” phase penetration because of occlusion at least in part of the monoadduct and/or bisadduct by the varnish and/or enamel.

The coating can be removed from the nail plate after a sufficient time has passed to allow the nail to reform to a contiguous, natural nail plate. In some instances, the coating will naturally wear away as the nail plate regrows. If early removal is desired, the coating can be eliminated by solvent and abrasion. Film former coatings can also be removed by solvent elimination.

### Functional Properties of Adducts on Nails

The tests for strengthening and adduct development described below show that the Michael addition compounds deliver better nail strengthening than does use of the diamine alone. Michael addition compounds include those with simple diamines as described in Applicant's PCT application No. WO/US23/65266, the disclosure of which is incorporated herein by reference and those with complex diamine described herein. It is believed that the use of complex organodiamines for the adducts as well as inclusion of optional residues and unreacted starting material as opposed to simple alkyl diamines may enable use of higher concentrations of adducts in medium and better treatment of nail brittleness, breakage and injury. The aqueous solubility of the complex organodiamine adducts and the corresponding unreacted diamines is influenced by the kind and number of hydrophilic groups of the complex organodiamine moiety in the adduct and the unreacted starting material. Although the chemical, electronic, polar, hydrophilic/hydrophobic factors managing nail penetration are complex, it is believed that the interactions between these functions and the mono and bis adducts having the complex organodiamine moieties can lead to significant improvements in treatment of nail malconditions.

### Preparation of Adducts

The Michael adducts are prepared by any of three methods: a boiling water technique; a boiling aqueous alcoholic technique optionally with neutral to alkaline pH control; and an “at rest” neat or alcoholic technique with optional neutral to alkaline pH control.

Using the boiling water technique, the carboxylic acid monoadduct may be prepared following the procedure outlined in DE848045 the disclosure of which is incorporated herein by reference. One equivalent of maleic anhydride in distilled water may be heated to about 100° C. and thereafter 2 equivalents of isophorone diamine or diaminocyclohexane or bis(3-aminopropyl) diethylene glycol may be added and the reaction mixture stirred at about 100° C. for 24 hours. Thereafter, about 2 equivalents of barium hydroxide may be added and heating continued for 48 hours. The hydroxide treatment hydrolyzes the anhydride group to carboxylate groups. The reaction mixture can be cooled to produce a barium salt precipitate which may be filtered and repeatedly washed with distilled water. This acid workup will convert the barium salts precipitate to barium sulfate and dissolve the monoadduct as the carboxylic acid monoadduct (along with a minor amount of the bisadduct). The suspension may be filtered, and the filtrate concentrated under reduced pressure to produce the carboxylic acid monoadduct as the sulfuric acid salt. Purification by precipitation from a water/methanol solution, and concentration of the filtrate under reduced pressure can yield the purified carboxylic acid monoadduct as the sulfuric acid salt.

The methyl ester monoadduct may be prepared in a similar manner or by esterifying the carboxylic acid monoadduct.

Using the alcoholic or alcoholic neutral to alkaline pH technique, the preparation of the methyl ester monoadduct may be conducted by forming a solution of 1 molar equivalent of dimethyl maleate (liquid) in a solvent system of methanol, ethanol, propanol, butanol and/or monoglyme and/or diethylene glycol monomethyl ether, and preferably methanol, and optionally with a 1 wt % to 3 wt % water and heating the solution to near boiling as described by the procedures given in U.S. Pat. Nos. 3,158,635; 5,846,925 and 6,465,690, the disclosures of which are incorporated herein by reference. A solution of 2 molar equivalents of isophorone diamine or diaminocyclohexane or bis(3-aminopropyl) diethylene glycol in the same solvent system may be added and the solution maintained at near boiling for at least a day. Additionally, the conditions of the alcoholic technique can be adjusted to neutral or slightly alkaline (up to about pH 9 or 10). The reaction mixture may be thereafter cooled and chromatographed to obtain the dimethyl ester monoadduct.

The bisadduct may be prepared in a similar manner by changing the molar equivalent ratio from 1:2 dimethyl maleate to the diamine to a 2:1 molar equivalent ratio. The molar excess of dimethyl maleate will promote the Michael addition of each amine group of the diaminohexane to separate dimethyl maleate molecules.

Alternatively, the carboxylic acid embodiments of the monoadduct and the bisadduct may be converted to the methyl ester embodiments by methylation of the carboxylic acid group with azomethane or with methyl iodide.

Using the “at rest” technique will also produce the Michael addition products. The starting materials at a ratio of maleic acid to diamine calculated to provide the monoadduct or bisadduct or to produce the monoadduct/bisadduct mixture with an excess of unreacted diamine are dissolved in alcoholic solvent with no water or in alcoholic solvent with base providing a manipulated pH of from 6 to 9 and allowed to remain at rest at ambient condition in a closed flask for up to six to twelve months.

Using the accelerated “at rest” technique, the alcoholic solution or the manipulated pH alcoholic solution be warmed at a temperature from 40 to 60° C., preferably up to 50° C., for an interim period of one to two months at the start of the process.

At the end of either the at rest process or the accelerated at rest technique, the alcoholic solution can be chromatographed to isolate the Michael addition products, or to remove only the unreacted maleic acid or alternatively used without further purification as a treatment composition. Preferably, removal of the maleic acid and use of the resulting alcoholic solution can be as a treatment composition. Liquid chromatography-mass spec analysis of the contents of the at rest alcoholic solution (no base) shows that from 0.5 wt % to 10 wt % of the combined monoadduct and bisadduct are produced depending upon whether interim heating of the solution is provided. With use of a basic alcoholic accelerated technique, the combined monoadduct and bisadduct amount can be higher such as from 10 wt % to 50-70 wt % wherein the wt %'s are relative to the total amount of mono/bisadduct and unreacted starting material and reaction residues.

### Preparation of Treatment Composition

A treatment composition for application to a nail plate may be prepared by combining the monoadduct and/or bisadduct and/or the adducts with the optional starting material residue(s) and unreacted starting material(s) in an aqueous or organo-aqueous medium. The carboxylic acid monoadduct and/or bisadduct embodiment may be appropriately formulated as a strongly strengthening treatment composition by adjusting the pH of the aqueous or organo-aqueous medium to enable these carboxylic acid embodiments to be presented as electrically neutral zwitterionic embodiments. While a pH ranging from highly acidic to highly basic will provide a suitable treatment composition, pH adjustment may be made to provide a most appropriate treatment composition by providing a pH of the aqueous or organo-aqueous medium of approximately 2 to 5, preferably 2.5 to 3.5. This adjustment may be obtained by combining the neutralized carboxylic acid mono and/or bis adduct embodiments with the medium to provide an appropriate pH or by combining the non-neutralized forms of the carboxylic acid mono and/or bis adduct embodiments with a medium already adjusted by addition of acid and optional acid buffer to have a pH of approximately 2.5 to 3.5. While a medium with a pH of from 2 to 10 may be employed to provide the treatment composition having at least some strengthening effect on nails, adjustment of the medium pH to 2.5 to 3.5 may be employed to provide a stronger strengthening effect.

The methyl ester monoadduct and/or bisadduct as well as those with the optional starting material residue(s) and unreacted starting material(s) embodiments may be appropriately formulated in aqueous or organo-aqueous medium at a pH of rom 2 to 11 to provide at least some nail strengthening effect. By adjusting the pH of the medium to have a basic pH of from 6 to 10, preferably 7.5 to 9.5, the treatment composition with the methyl ester monoadduct and/or bisadduct as well as the optional starting material residue(s) and unreacted starting material(s) may provide a stronger strengthening effect. This pH adjustment may be accomplished through use of a base such as sodium hydroxide and preferably in combination with a basic buffer. The basic pH may range from about 8.9 to 9.5.

The medium before pH adjustment may be distilled water or an organo-aqueous medium of no more than 50 volumetric percent of, preferably no more than 30 volumetric percent, more preferably no more than 20 volumetric percent of methanol, ethanol or isopropanol or a mixture thereof.

### Nail Strengthening Tests

The nail strengthening tests examine the ability of the mono and/or bisadduct to strengthen the nail plate. The tests determine an ability of a point probe under increasing pressure to pierce or otherwise break a human fingernail. The human fingernails are obtained as nail clippings of extended natural fingernails. The multiple nail clippings obtained have an average cross-sectional size of from 0.41 to 0.43 mm, a length of about 5 mm and a width of about 8 to 9 mm.

The nail clippings may be cleaned by immersing in isopropanol optionally with ethyl acetate to remove any residual nail varnish, washed with water and then coated with a sample of the adduct test solution and allowed to dry. A sample coated clipping prepared in this manner may be placed in a custom holder of a texture strength determination machine. The holder unit clamped the entire coated clipping on top of a metal base so that the underside of the coated clipping rested against the metal base. A needle probe affixed to an automated piston powered by the machine may be positioned on top of the coated clipping so that the needle tip rested just appurtenant to the surface of the coated nail clipping. The machine may be actuated to begin pressing the needle probe onto the coated nail clipping at a digitally increasing pressure. The pressure at which the coating nail clipping can be pierced by the needle probe can be recorded as the coated nail hardness.

### Protocol for Nail Strength Determination

The protocol for operation of the TA.XTplus connect textural analyzer by Texture Technologies Corp., Hamilton MA used to perform the puncture tests follows the procedure outlined by Texture Technologies and as guided and operated by a software program for conducting a variable controlled needle puncture test. This operating program for the controlled pressure puncture test on the TA.XTplus (Texture Technologies Corp.) analyzer was developed by Exponent software (Stable Micro Systems, Ltd). Nail hardness was measured as the peak force throughout the puncture test. The percentage difference of treated and control nail hardness was used to determine the efficacy of embodiments of the nail treatment composition according to the invention.

### Preparation of Treatment Composition Aliquots

To conduct the nail puncture test with nail clippings coated with the Treatment Composition according to the invention, exemplary aliquots of the Treatment Composition with the monoadduct mixture at different concentrations may be prepared.

### Nail Clipping Preparation

The nail clipping substrates can be collected by clipping as single pieces the free distal ends of the five fingernails of volunteers.

Each fingernail clipping may be cut into two halves then cleaned with lint-free wipes soaked in iso-propanol to remove any surface residue. The halves may be paired and allowed to dry overnight under ambient conditions. One half of the pair may be coated with the treatment composition according to the following procedure. The other half can remain uncoated and may be used as the comparative null puncture test piece so that the extent of strengthening by the treatment composition could be accurately determined.

### Strengthening Efficacy and Adduct Development

A comparison of the strengthening efficacy of any diamine alone (example hexane diamine) and maleic acid alone compared with any mono Michael adduct (example of purified monoadduct of hexane diamine and maleic acid) is shown on Table 1.

TABLE 1
Michael Bisadduct and Hexmethylene diamine comparison

| | Average | | |
|---|---|---|---|
| | Puncture Harness | | |
| Formula | % Increase | STDEV | N |
| 1.67% hexamethylene diamine | 25.86 | 7.58 | 3 |
| (HMDA) in ethanol | | | |
| 3.34% Maleic acid (MA) in | 12.83 | 9.09 | 3 |
| ethanol | | | |
| 5% Michael bisadduct of | 42.59 | 18.75 | 6 |
| HMDA/MA at a 1:2 ratio in ethanol | | | |
| (2X per day for 6 days) | | | |

The strengthening efficacy of the at rest Michael adduct prepared by the at rest procedure is conducted with maleic acid (MA) and hexamethylene diamine (HMDA) in absolute ethanol or in 95% ethanol held at ambient temperature for 6 months. A second at rest sample is prepared by combining the same starting materials and solvent and holding the mixture at 45° C. for 2 months and then at ambient temperature for 4 months. The graph shows that aging for 6 months at ambient temperature produced about 0.5 wt % of the combined adducts and aging at 45° C. for 2 months followed by aging for 4 months at ambient temperature produced about 10 wt % combined adducts.

A higher percentage development of the combined adducts by the at rest procedure can be obtained by maintaining the mixture at an elevated temperature of about 45° C. to 55° C. for a six month period. The yield of combined adducts by the at rest procedure can also be increased by providing a pH of the mixture of about 5 to 9, preferably 5.5 to 8. A management of pH enables at least a portion of the diamine to be unprotonated which facilitates nucleophilic Michael addition, However, maintenance of high pH such as at 10 or 11 or higher will tend to convert all of the maleic acid into the salt form and decrease Michael addition efficiency because of the resulting double anionic character of the maleic acid salt form.

A graph showing the at rest development of the adducts produced in an alcohol mixture of starting materials at rest at ambient temperature for 6 months is provided by Table 2.

A combination of the monoadduct, the bisadduct, the diamine and the maleic acid is prepared by combining the maleic acid, the diamine neat in a closed container such as a reaction flask at the desired weight ratio to dissolve the maleic acid in the diamine and form a liquid solution or dispersion (hereinafter mixture). The mixture is allowed to rest at ambient temperature for a period of from at least two months to as much as six months.

The weight percentage of combined adducts in the rested mixture can be increased by allowing the mixture to rest at elevated temperature such as from 40° C. to 60° C. for the period mentioned above. At elevated temperature, the amount of the combined adducts may be up to about 40 to 70 weight percent of the total weight of the mixture.

The weight percentage of the combined adducts in the rested mixture may also be increased by addition of a solvent such as an aqueous alcohol such as 85 to 95 percent ethanol in water and a base such as sodium or potassium hydroxide to provide a pH of about 9 to 11. With the addition of solvent and base, the amount of combined adducts may be increased to 30 to 80 weight percent of the total weight of the mixture.

Depending upon the molar ratio of diamine to maleic acid, the remainder molar amount of diamine will be the same as the remainer molar amount or less than the remainder molar amount of maleic acid. In particular, if the molar ratio of maleic acid to diamine is 2 or higher, the Michael addition will primarily form the bisadduct. This kind of high molar ratio will therefore reduce the remaining molar amount of diamine to less than expected when the Michael addition is incomplete.

Following the foregoing rest mixture technique, ethanolic mixtures of diamine with maleic acid (3.34 wt %. 0.0288 moles) and the following diamines were prepared:
a) hexamethylene diamine (1.16 wt %, 0.01 moles);
b) isophorone diamine (2.43%, 0.0143 moles);
c) bis (3-aminopropyl) diethylene glycol (3.76 wt %, 0.0171 moles).

The molar amounts for the maleic acid and diamine provided slightly more than 2 moles of maleic acid per 1 mole of diamine (hexane diamine and isophorone diamine) and slightly less than 2 moles of maleic acid per 1 mole of diamine (bis(3-aminopropyl) diethylene glycol. As explained above, use of the ambient temperature rest procedure for 6 months with these above described molar amounts produced essentially all bisadduct for the first two diamines (a and b) and a mixture of a lesser amount of monoadduct and a greater amount of bisadduct for the third diamine (c).

The resulting rested mixture was also found to contain the unreacted maleic acid and diamine as well as the Michael addition mixture. Overall, the relative molar concentration of the sum of bisadduct and monoadduct relative to the total molar amount of maleic acid and diamine used as starting materials is approximately 20% +5% as determined by LC-MS study of the mixture.

These mixtures (a, b and c) were used as treatment compositions in the above described nail strengthening procedure. Treatment was conducted two times per day for six days. The designator “n” indicates the number of nail clippings treated for the six day period. Results are tabularized as Table 3. Because the puncture tests are conducted with nail clippings from humans whose nails are not standard and uniform in thickness, tensile strength and biochemical makeup, the results provided in Table 1 compared with the result of Table 3 C1 differ as a result of these different individual humans' fingernail characteristics. However, the results of Table 3 C1-C3 are directly comparable because of the use of aliquots of the same human fingernail clipping for all three Examples C1-C3.

TABLE 3
Example C1, n = 20

| | |
|---|---|
| Maleic Acid | 3.34% |
| Hexamethylene Diamine | 1.16% |
| producing bisadduct | |
| Ethanol | 95% |
| Average Nail Hardness | 11.55% |
| Increment | |

Example C2, n=10

| | |
|---|---|
| Maleic Acid | 3.34% |
| Isophorone Diamine | |
| producing bisadduct | 2.43% |
| Ethanol | 94.23% |
| Average Nail Hardness | 17.87% |
| Increment | |

Example C3, n=9 (lost one sample during treatment)

| | |
|---|---|
| Maleic Acid | 3.34% |
| Bis(3- | 3.76% |
| aminopropyl)diethylene | |
| glycol producing major | |
| amount of bisadduct and | |
| minor amount of | |
| monoadduct | |
| Ethanol | 92.90% |
| Average Nail Hardness | 15.57% |
| Increment | |

The increase in average nail hardness for the complex diamines relative to the alkyl diamine demonstrates that the use of complex diamines with their contribution to lipophilicity of the mixture improves the nail hardening ability of the mixture. This improvement also demonstrates that the purified monoadduct and bisadduct of complex diamines will show improvement in nail hardening ability.

### Statement of Embodiments

The following statements describe embodiments of the invention according to the foregoing description and experimental details. These statements provide further disclosure of these aspects, features and parameters and may serve as claims of the invention.

### Statements

1. A method for treatment of a damaged nail plate comprising application to the damaged nail plate, a treatment composition comprising an organic or aqueous or aqueous-organic medium in combination with a monoadduct of any one or more of Formulas VI, IXA, IXB, XIIIA and XIIIB and/or its amine and/or carboxyl salt; or with a bisadduct of any one or more of Formulas VII, XIA, XIB, XIVA and XIVB and/or its carboxyl salt; or with a mixture of the monoadduct and bisadduct of any one or more of Formulas VI and VII or Formulas IXA and XIA or Formulas IXB and XIB or Formulas XIIIA and XIVA or Formulas XIIIB and XIVB and/or their amine and/or carboxyl salts:

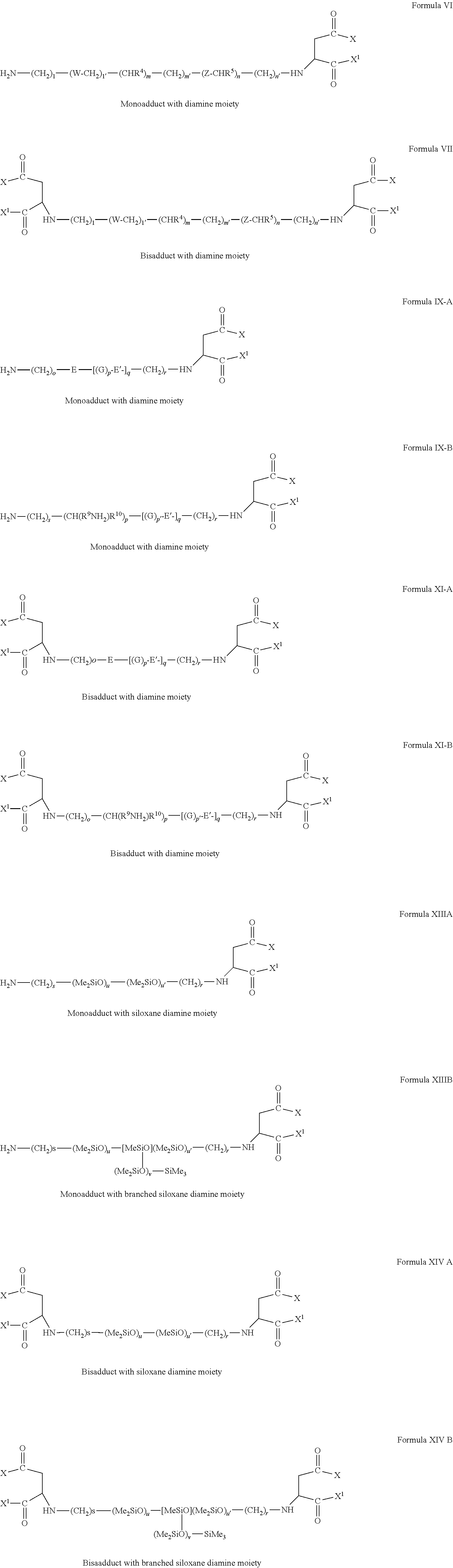

### Wherein:

the designators k, l, l′, m, m′, n, n′, o, p, q, r and s and substituents X, X′ W, Z E, E′ and G are defined to provide:
1) l, l′, m, m′, n and n′ each independently may be zero or an integer of from 1 to 100, and the sum of l, l′, m, m′, n and n′ may be at least 2 and as much as 100, preferably up to 50, more preferably up to 30, even more preferably up to 20 with individual sums being 2, 3, 4, 5, 6, 7, 8, 9, 10, 11, 12, 13, 15 or 15;
2) Both of X and X¹ may be —OH, —OR² or —NHR³;
3) W and Z each independently may be O, S or NR⁶;
4) R² is C1-C4 linear alkyl;
5) R³ is H, C1-C4 linear alkyl,
6) R⁴ and R⁵ each individually and separately may be hydrogen or C1-C10 alkyl optionally substituted by one or more of a C1-C4 alkyl group, a C1-C4 alkoxy group, a C1-C4 alkthio group, a C1-C4 alkyl substituted by —CO₂—C1-C4 alkyl group, a C1-C4 alkyl substituted by —CONH—C1-C4 alkyl group, a C1-C4 alkyl substituted by —SO₂—C1-C4 alkyl group, and a C1-C4 alkyl substituted by —SO₂—NH—C1-C4 alkyl group;
7) R⁶ may be hydrogen or a C1-C20 linear or branched alkyl group;
8) E may be cyclopentyl, cyclohexyl, C1-C4 mono, di or trialkyl cyclohexyl, phenyl or C1-C4 alkyl phenyl, C6-C10 aryl with 1 or 2 rings or C1-C4 mono- or multi-alkyl C6-C10 aryl with 1 or 2 rings, pyridinyl, 1,3,5-triazinyl or alkyl- or phenyl-substituted 1,3,5 triazine;
9) E′ may be cyclopentyl or cyclohexyl, C1-C4 mono, di or trialkyl cyclohexyl, phenyl or C1-C4 alkyl phenyl;
10) G may be —(CH₂)ₖ—, —SO₂—, —NH;
11) k may be an integer of from 1 to 10;
12) o and r each independently may be zero or an integer of from 1 to 4;
13) p, p′ and q each independently may be zero or 1;
14) s may be an integer of from 1 to 4;
15) u, u′ and v each independently may be zero or an integer of from 1 to 50, preferably 1-20, more preferably 1-10, most preferably 1-5;
16) R⁹ may be hydrogen or —(CH₂)ₖ—;
17) R¹⁰ may be hydrogen or a C1-C6 alkyl; and
the concentration of any one of the monoadducts or any one of the bisadducts or any one of the mixtures is at least 0.2 wt % relative to the total weight of the composition.
2. The method according to statement 1 wherein X and X¹ are both OH or X and X¹ together are anhydride —CO—O—CO—.
3. The method according to statement 2 wherein X and X¹ are both OH.
4. The method according to statement 1 wherein X and X¹ are both —OR² and R² is methyl or ethyl, preferably methyl.
5. The method according to any of the preceding statements comprising substantially only the monoadduct of any one or more of Formulas VI, IXA, IXB, XIIIA or XIIIB.
6. The method according to statement 5 comprising only the monoadduct.
7. The method according to any of the preceding statements 1-4 comprising substantially only the bisadduct of any one or more of Formulas VII, XIA, XIB, XIVA or XIVB.
8. The method according to statement 7 comprising only the bisadduct.
9. The method according to any of the preceding statements 1-4 comprising substantially only the mixture of the monoadduct and bisadduct of one or more of Formulas VI and VII or Formulas IXA and XIA or Formulas IXB and XIB or Formulas XIIIA and XIVA or Formulas XIIIB and XIVB.
10. The method according to statement 9 comprising only the mixture.
11. The method according to any of statements 9, 10 wherein the wt percentage of the bisadduct present in the mixture is no more than about 40 mol percent, preferably no more than about 20 mol percent, more preferably no more than about 10 mol percent, especially more preferably no more than about 5 mol percent, most preferably no more than about 1 to 2 mol percent relative to the total weight of the mixture.
12. The method according to any of the preceding statements wherein the concentration of the monoadduct of any one or more of Formulas VI, IXA, IXB, XIIIA or XIIIB; or the bisadduct of any one or more of Formulas VII, XIA, XIB, XIVA or XIVB; or the mixture of the mono and bisadduct of any one or more Formulas VI and VII or Formulas IXA and XIA or Formulas IXB and XIB or Formulas XIIIA and XIVA or Formulas XIIIB and XIVB in the organic or aqueous or aqueous organic medium comprises from about 0.2 wt % to about 20 wt %, preferably from about 0.5 wt % to about 15 wt %, more preferably about 0.5 wt % to about 10 wt %, most preferably about 0.5 wt % to about 6.5 wt % relative to the total weight of the composition.
13. The method according to any of the preceding statements 1-6 wherein substantially only the monoadduct of any one or more of Formulas VI, IXA, IXB, XIIIA or XIIIB is present and its concentration in the organic, aqueous or aqueous organic medium comprises from about 1.5 wt % to about 10 wt %.
14. The method according to any of the preceding statements wherein the monoadduct and/or bisadduct with X and X′ as OH comprises an amine neutralized form as a hydrochloric, sulfuric, nitric and/or phosphoric acid salt and/or the carboxyl neutralized form as the alkali metal or ammonia base salt.
15. The method according to any of the preceding statements wherein the monoadduct of any one of Formulas VI, IXA, IXB, XIIIA or XIIIB and/or the bisadduct of any one of Formulas VII, XIA, XIB, XIVA or XIVB has X and X¹ as OH and the pH of the medium is about 1 to about 11, preferably about 2 to about 10, more preferably about 2 to about 7, especially more preferably about 2 to about 5, most preferably about 2.5 to about 3.5.
16. The method according to any of the preceding statements wherein the monoadduct of any one of Formulas VI, IXA, IXB, XIIIA or XIIIB and/or the bisadduct of any one of Formulas VII, XIA, XIB, XIVA or XIVB is a methyl ester and the pH of the medium is about 2 to about 11, preferably about 4 to about 10.5, more preferably about 6 to about 10, especially more preferably about 7.5 to about 10, most preferably about 7.8 to about 9.5.
17. The method according to any of the preceding statements wherein the medium is an aqueous medium.
18. The method according to any of preceding statements wherein the medium is an organic or aqueous organic medium and the organic is an alcohol selected from one or more of C1-C4 mono alcohol, a C2-C4 di alcohol or a C3-C4 tri alcohol or is a combination of the alcohol with a non-alcohol selected from one or more of a ketone, ester, acetate, dimethyl isosorbide, DMSO, DMF, THF.
19. The method according to statement 18 wherein the aqueous organic medium is aqueous alcohol, preferably aqueous ethanol medium.
20. The method according to statement 18 or 19 wherein the volume % (v %) of organic in the aqueous organic medium is in a range of from about 2 v % to about 50 v %, preferably about 2 v % to about 25 v %, more preferably about 2 v % to about 10 v % relative to the total volume of the medium.
21. The method according to any of the preceding statements wherein the monoadduct and/or the bisadduct are in un-neutralized and/or zwitterionic form and the medium is the aqueous organic medium with no more than 50 v % organic compound, preferably no more than 25 v % organic compound and the organic compound is an alcohol with no more than 2 v % of non-alcohol, preferably no more than 1 v % relative to the total volume of organic compound.
22. The method according to any of the preceding statements wherein the treatment composition further comprises at least one of a film forming polymer, a fatty alcohol, a rheologic agent, a nonionic surfactant, a penetration agent, a perfume, a preservative, a colorant or any combination thereof.
23. The method according to statement 22 comprising the rheologic agent and the penetration agent.
24. The method according to statement 22 comprising the rheologic agent, the penetration agent, the nonionic surfactant and optional film former polymer selected from a (meth)acrylate polymer, a (meth)acrylate-vinyl copolymer, an ester polymer, an amide polymer, a vinyl or olefin polymer, a urethane polymer or a cellulosic polymer.
25. A method for treatment of a damaged nail plate according to any of the preceding statements wherein the treatment composition further comprises at least a diamine having one or more of Formulas V, VIIIA, VIIIB, XIIA, XIIB wherein the diamine corresponds to the diamine moiety of the monoadduct or bisadduct or mixture thereof, and the treatment composition further comprises optionally a corresponding unsaturated carboxyl compound starting material of Formula IV:

H₂N—(CH₂)ₗ—(W—CH₂)_{l′}—(CHR⁴)ₘ—(CH₂)_{m′}—(Z—CHR⁵)ₙ—(CH₂)_{n′}—NH₂ Formula V

H₂N—(CH₂)ₒ-E-[(G)ₚ-E′-]_{q}-(CH₂)ᵣ—NH₂ Formula VIIIA

H₂N—(CH₂)ₒ—(CH(R⁹NH₂)R¹⁰)ₚ-[(G)_{p′}-E′-]_{q}-(CH₂)ᵣ—NH₂ Formula VIIIB

H₂N—(CH₂)ₛ-(Me₂SiO)ᵤ-(Me₂SiO)_{u′}—(CH₂)ᵣ—NH₂ Formula XIIA

H₂N—(CH₂)ₛ-(Me₂SiO)ᵤ-[MeSiO(SiMe₂O)ᵥ—SiMe₃)](Me₂SiO)_{u′}—(CH₂)ᵣ—NH₂ Formula XIIB

X—OC—HC═CH—CO—X; Formula IV
wherein the designators k, l, l′, m, m′, n, n′, o, p, q, r and s and substituents X, X′ W, Z E, E′ and G are defined in statement 1; or,
   both instances of X and X together with their CO groups form an anhydride of the formula —C═O—O—C═O—, or an imide of the formula —C═O—NR³—C═O.
26. The method according to statement 25 wherein the mixture of the monoadduct and bisadduct is present along with the corresponding diamine or diamines of Formulas V, VIIIA, VIIIB, XIIA and XIIB and the optional unsaturated carboxyl compound of Formula IV.
27. The method according to statement 25 or 26 wherein the molar ratio of the diamine of one or more of Formulas V, VIIIA, VIIIB, XIIA and/or XIIB relative to the corresponding monoadduct or corresponding bisadduct or corresponding mixture of monoadduct and bisadduct is at least 1:1.
28. The method according to statement 27 wherein the molar ratio is at least 2:1.
29. The method according to statement 27 wherein the molar ratio is at least 3:1.
30. The method according to statement 27 wherein the molar ratio is from about 1:1 to about 3:1.
31. The method according to statement 25 wherein the wt % amount of diamine of any one or more of Formulas V, VIIIA, VIIIB, XIIA, XIIB and optional unsaturated carboxyl compound of Formula IV is no more than about 95 wt % of the wt amount of the corresponding monoadduct, corresponding bisadduct or corresponding mixture of monoadduct and bisadduct.
32. The method according to statement 31 wherein the wt % amount is no more than about 50 wt %, preferably no more than about 25 wt %, more preferably no more than about 10 wt %.
33. The method according to any of statements 25-32 wherein the treatment composition is manipulated before application to damaged nail plate to remove diamine of any of Formulas V, VIIIA, VIIIB, XIIA, XIIB and unsaturated carboxyl compound of Formula IV.
34. The method according to statement 33 wherein the treatment composition is manipulated before application to the damaged nail plate to separate the bisadduct, separate the monoadduct, separate the diamine and separate the unsaturated carboxyl compound and include the monoadduct and/or the bisadduct and/or the mixture thereof in the treatment composition to be applied to the damaged nail.
35. The method according to statement 34 wherein the separated bisadduct, separated monoadduct and separated diamine are isolated and purified.
36. A method for treating the damaged nail plate of any of statements 1-35 comprising applying the treatment composition one or more times to the damaged nail a composition.
37. The method according to any of statements 1-36 wherein the treatment composition is applied at least once with a brush, sponge, felt or cloth.
38. The method according to statement 36 or 37 wherein the composition is applied at least 4 times to the nail.
39. The method according to statement 38 wherein the treatment composition used as the final application further comprises a film forming polymer.
40. Use of the treatment composition of any of the preceding claims as a treatment for damaged nails.
41. A treatment composition comprising a monoadduct of any one or more of Formulas VI, IXA, IXB, XIIIA and XIIIB and/or its amine and/or carboxyl salt of statement 1; or a bisadduct of any one or more of Formulas VII, XIA, XIB, XIVA and XIVB and/or its carboxyl salt of statement 1; or a mixture of the monoadduct and bisadduct of any one or more of Formulas VI and VII or Formulas IXA and XIA or Formulas IXB and XIB or Formulas XIIIA and XIVA or Formulas XIIIB and XIVB and/or their amine and/or carboxyl salts of statement 1, or a combination of the monoadduct or the bisadduct or their mixture with at least a diamine having one or more of Formulas V, VIIIA, VIIIB, XIIA, XIIB of statement 25 wherein the diamine corresponds to the diamine moiety of the monoadduct or bisadduct or mixture thereof, and optionally a corresponding unsaturated carboxyl compound starting material of Formula IV.

### Summary Statements

The inventions, examples and results described and claimed herein may have attributes and embodiments include, but not limited to, those set forth or described or referenced in this application.

All patents, publications, scientific articles, web sites and other documents and ministerial references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the scope of the invention. Other aspects, advantages, and modifications are within the scope of the following claims and the present invention is not limited except as by the appended claims.

1. A method for treatment of a damaged nail plate comprising administering to the nail plate a treatment composition comprising an organic or aqueous or aqueous-organic medium in combination with a monoadduct of any one or more of Formulas VI, IXA, IXB, XIIIA and XIIIB and/or its amine and/or carboxyl salt; or with a bisadduct of any one or more of Formulas VII, XIA, XIB, XIVA and XIVB and/or its carboxyl salt; or with a mixture of the monoadduct and bisadduct of any one or more of Formulas VI and VII or Formulas IXA and XIA or Formulas IXB and XIB or Formulas XIIIA and XIVA or Formulas XIIIB and XIVB and/or their amine and/or carboxyl salts: 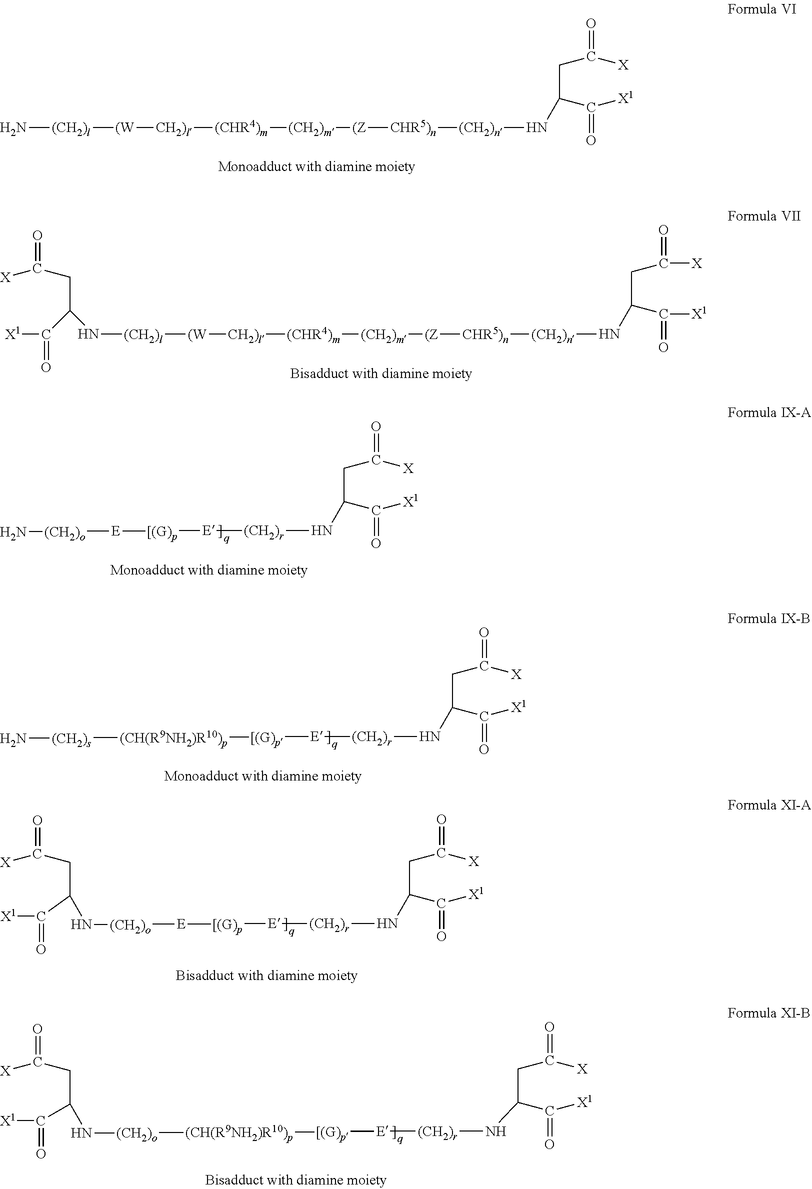 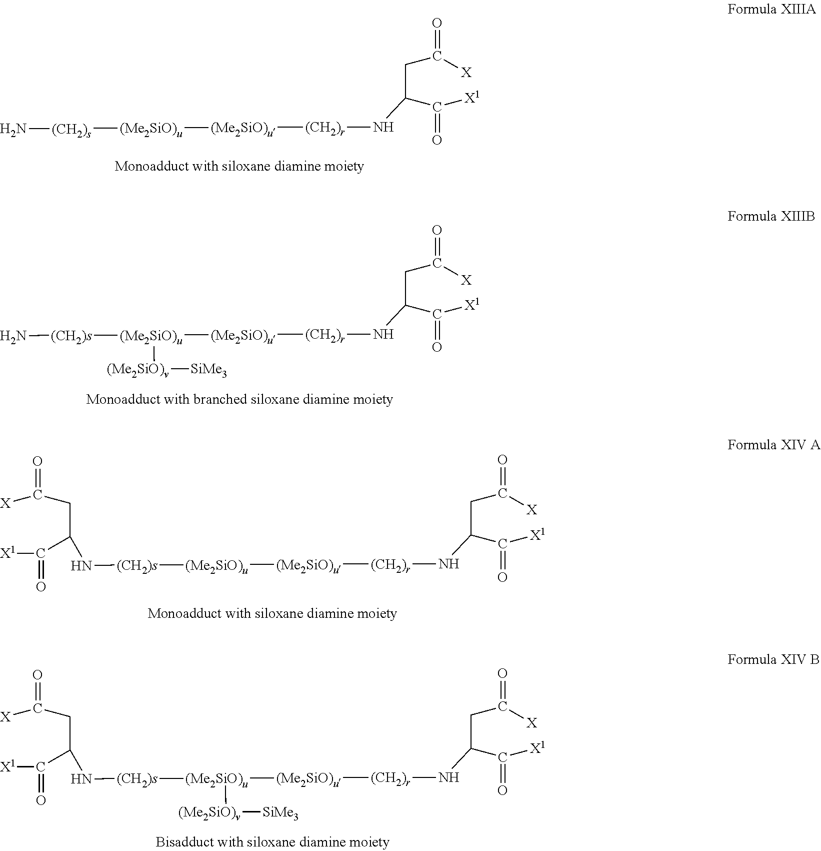
wherein:
the designators k, l, l′, m, m′, n, n′, o, p, q, r and s and substituents X, X′ W, Z E, E′ and G are defined to provide:
1) l, l′, m, m′, n and n′ each independently may be zero or an integer of from 1 to 100, and the sum of l, l′, m, m′, n and n′ may be at least 2 and as much as 100, preferably up to 50, more preferably up to 30, even more preferably up to 20 with individual sums being 2, 3, 4, 5, 6, 7, 8, 9, 10, 11, 12, 13, 15 or 15;
2) Both of X and X¹ may be —OH, —OR² or —NHR³;
3) W and Z each independently may be O, S or NR⁶;
4) R² is C1-C4 linear alkyl;
5) R³ is H, C1-C4 linear alkyl,
6) R⁴ and R⁵ each individually and separately may be hydrogen or C1-C10 alkyl optionally substituted by one or more of a C1-C4 alkyl group, a C1-C4 alkoxy group, a C1-C4 alkthio group, a C1-C4 alkyl substituted by —CO₂—C1-C4 alkyl group, a C1-C4 alkyl substituted by —CONH—C1-C4 alkyl group, a C1-C4 alkyl substituted by —SO₂—C1-C4 alkyl group, and a C1-C4 alkyl substituted by —SO₂—NH—C1-C4 alkyl group;
7) R⁶ may be hydrogen or a C1-C20 linear or branched alkyl group;
8) E may be cyclopentyl, cyclohexyl, C1-C4 mono, di or trialkyl cyclohexyl, phenyl or C1-C4 alkyl phenyl, C6-C10 aryl with 1 or 2 rings or C1-C4 mono- or multi-alkyl C6-C10 aryl with 1 or 2 rings, pyridinyl, 1,3,5-triazinyl or alkyl- or phenyl-substituted 1,3,5 triazine;
9) E′ may be cyclopentyl or cyclohexyl, C1-C4 mono, di or trialkyl cyclohexyl, phenyl or C1-C4 alkyl phenyl;
10) G may be —(CH₂)ₖ—, —SO₂—, —NH;
11) k may be an integer of from 1 to 10;
12) o and r each independently may be zero or an integer of from 1 to 4;
13) p, p′ and q each independently may be zero or 1;
14) s may be an integer of from 1 to 4;
15) u, u′ and v each independently may be zero or an integer of from 1 to 50, preferably 1-20, more preferably 1-10, most preferably 1-5;
16) R⁹ may be hydrogen or —(CH₂)ₖ—;
17) R¹⁰ may be hydrogen or a C1-C6 alkyl.

2. The method according to claim 1 wherein the treatment composition further comprises a diamine of one or more of Formulas V, VIIIA, VIIIB, XIIA and XIIB corresponding to the diamine moiety of the monoadduct of one or more of Formula VI, IXA, IXB, XIIIA and XIIIB; or a diamine of one or more of Formulas V, VIIIA, VIIIB, XIIA and XIIB corresponding to the diamine moiety of the bisadduct of any one or more of Formulas VII, XIA, XIB, XIVA and XIVB; or a diamine of one or more of Formulas V, VIIIA, VIIIB, XIIA and XIIB corresponding to a diamine moiety of a mixture of monoadduct and bisadduct of any one or more of Formulas VI and VII or Formulas IXA and XIA or Formulas IXB and XIB or Formulas XIIIA and XIVA or Formulas XIIIB and XIVB; and optionally an unsaturated carboxyl compound of Formula IV:
H₂N—(CH₂)ₗ—(W—CH₂)_{l′}—(CHR⁴)ₘ—(CH₂)_{m′}—(Z—CHR⁵)ₙ—(CH₂)_{n′}—NH₂ Formula V
H₂N—(CH₂)ₒ-E-[(G)ₚ-E′-]_{q}-(CH₂)ᵣ—NH₂ Formula VIIIA
H₂N—(CH₂)ₒ—(CH(R⁹NH₂)R¹⁰)ₚ-[(G)_{p′}-E′-]_{q}-(CH₂)ᵣ—NH₂ Formula VIIIB
H₂N—(CH₂)ₛ-(Me₂SiO)ᵤ-(Me₂SiO)_{u′}—(CH₂)ᵣ—NH₂ Formula XIIA
H₂N—(CH₂)ₛ-(Me₂SiO)ᵤ-[MeSiO(SiMe₂O)ᵥ—SiMe₃)](Me₂SiO)_{u′}—(CH₂)ᵣ—NH₂ Formula XIIB
X—OC—HC═CH—CO—X; Formula IV
wherein the designators k, l, l′, m, m′, n, n′, o, p, q, r and s and substituents X, X′ W, Z E, E′ and G are defined as provided by claim 1.

3. The method according to preceding claim 2 wherein the treatment composition comprises only the monoadduct of any one or more of Formulas VI, IXA, IXB, XIIIA or XIIIB in combination with the diamine of one or more of Formulas V, VIIIA, VIIIB, XIIA and XIIB corresponding to the diamine moiety or moieties of the monoadduct.

4. The method according to claim 2 wherein the treatment composition comprises only the bisadduct of any one or more of Formulas VII, XIA, XIB, XIVA or XIVB in combination with the diamine of one or more Formulas V, VIIIA, VIIIB, XIIA and XIIB corresponding to the diamine moiety or moieties of the bisadduct.

5. The method according to claim 2 wherein treatment composition comprises only the mixture of the monoadduct and bisadduct of one or more of Formulas VI and VII or Formulas IXA and XIA or Formulas IXB and XIB or Formulas XIIIA and XIVA or Formulas XIIIB and XIVB in combination with the diamine of one or more of Formulas V, VIIIA, VIIIB, XIIA and XIIB corresponding to the diamine moiety or moieties of the mixture.

6. The method according to claim 2 wherein the treatment composition is manipulated before application to the damaged nail plate to remove the diamine of one or more of Formulas V, VIIIA, VIIIB, XIIA and XIIB.

7. The method according to claim 1 wherein the concentration of the monoadduct or bisadduct or their mixture in the organic or aqueous or aqueous organic medium comprises of from about 0.2 wt % to about 20 wt % relative to the total weight of the composition, or is at least 0.2 wt % relative to the total weight of the composition.

8. The method according to claim 2 wherein the concentration of the monoadduct or bisadduct or the mixture in combination with diamine in the organic or aqueous or aqueous organic medium comprises of from about 0.2 wt % to about 20 wt % relative to the total weight of the composition.

9. The method according to claim 1 wherein the treatment composition comprises the monoadduct of any one or more of Formulas VI, IXA, IXB, XIIIA and XIIIB; or the bisadduct compound of any one or more of Formulas VII, XIA, XIB, XIVA and XIVB; or the mixture of the monoadduct and bisadduct of any one or more of Formulas VI and VII or Formulas IXA and XIA or Formulas IXB and XIB or Formulas XIIIA and XIVA or Formulas XIIIB and XIVB wherein has X and X¹ are OH and the medium has a pH of about 2 to about 7.

10. The method according to claim 1 wherein the medium is an aqueous medium or is an aqueous organic medium and the organic is an alcohol selected from one or more of C1-C4 mono alcohol, a C2-C4 di alcohol or a C3-C4 tri alcohol optionally in combination with a non-alcohol selected from one or more of a ketone, ester, acetate, dimethyl isosorbide, DMSO, DMF, THF or any combination thereof.

11. The method according to claim 10 wherein the volume % (v %) of organic in the aqueous organic medium is in a range of from about 2 v % to about 95v %.

12. The method according to claim 1 wherein the treatment composition further comprises at least one of a film forming polymer, a fatty alcohol, a rheologic agent, a nonionic surfactant, a penetration agent, a perfume, a preservative, a colorant or any combination thereof.

13. The method according to claim wherein the treatment composition further comprises at least the rheologic agent and the penetration agent.

14. The method according to claim 12 wherein the film former polymer is selected from a (meth)acrylate polymer, a (meth)acrylate-vinyl copolymer, an ester polymer, an amide polymer, a vinyl or olefin polymer, a urethane polymer or a cellulosic polymer.

15. The method according to claim 1 wherein the treatment composition is prepared from a Michael addition reaction conducted in organic or aqueous or aqueous organic medium with a diamine of any one of Formulas V, VIIIA, VIIIB, XIIA, XIIB, and an unsaturated carboxyl compound of Formula IV to form one or more Michael addition products:
H₂N—(CH₂)ₗ—(W—CH₂)_{l′}—(CHR⁴)ₘ—(CH₂)_{m′}—(Z—CHR⁵)ₙ—(CH₂)_{n′}—NH₂ Formula V
H₂N—(CH₂)ₒ-E-[(G)ₚ-E′-]_{q}-(CH₂)ᵣ—NH₂ Formula VIIIA
H₂N—(CH₂)ₒ—(CH(R⁹NH₂)R¹⁰)ₚ-[(G)_{p′}-E′-]_{q}-(CH₂)ᵣ—NH₂ Formula VIIIB
H₂N—(CH₂)ₛ-(Me₂SiO)ᵤ-(Me₂SiO)_{u′}—(CH₂)ᵣ—NH₂ Formula XIIA
H₂N—(CH₂)ₛ-(Me₂SiO)ᵤ-[MeSiO(SiMe₂O)ᵥ—SiMe₃)](Me₂SiO)_{u′}—(CH₂)ᵣ—NH₂ Formula XIIB
X—OC—HC═CH—CO—X; Formula IV
wherein the designators k, l, l′, m, m′, n, n′, o, p, q, r and s and substituents X, X′ W, Z E, E′ and G are defined in claim 1; or both instances of X and X together with their CO groups form an anhydride of the formula —C═O—O—C═O—, or an imide of the formula —C═O—NR³—C═O—; and upon formation of the one or more Michael addition products, the X of the X—OC— adjacent to the unsaturated carbon residue to which the amine nitrogen is bonded becomes X¹; and
wherein
the one or more Michael addition products comprise:
a) the monoadduct of any one of Formula VI, IXA, IXB, XIIIA or XIIIB; or
b) the bisadduct of any one of Formula VII, XIA, XIB, XIVA or XIVB; or
c) the mixture of the monoadduct and bisadduct of Formulas VI and VII or Formulas IXA and XIA or Formulas IXB and XIB or Formulas XIIIA and XIVA or Formulas XIIIB and XIVB;
or the treatment composition comprises Michael addition product a) monoadduct, or Michael addition product b) bisadduct or Michael addition product c) mixture in combination with unreacted diamine of any one of Formulas V, VIIIA, VIIIB, XIIA, XIIB corresponding to the diamine moiety or moieties of the monoadduct or the bisadduct or the mixture and unreacted unsaturated carboxyl compound.

16. The method according to claim 15 wherein the Michael addition products are isolated and purified to remove unreacted diamine and unreacted unsaturated carboxyl compound.

17. The method for treating a damaged nail according to claim 1 comprising applying the treatment composition one or more times to the damaged nail.

18. The method according to claim 17 wherein the treatment composition is applied at least 4 times to the damaged nail.

19. The method according to claim 18 wherein the treatment composition includes at least one or more of a rheology agent, a penetration agent, a fatty alcohol, a colorant and a nonionic surfactant or any combination thereof and optionally the treatment composition used as the final application further comprises a film forming polymer.

20. (canceled)
